SEQUENCE LISTING (121 sequences)

SEQ ID NO: 1 - RNA, 21 nt, Artificial Sequence
  siRNA
ggugauuggucgaggagcunn

SEQ ID NO: 2 - RNA, 21 nt, Artificial Sequence
  siRNA
agcuccucgaccaaucaccnn

SEQ ID NO: 3 - RNA, 21 nt, Artificial Sequence
  siRNA
aauucugaaacgaugccccnn

SEQ ID NO: 4 - RNA, 21 nt, Artificial Sequence
  siRNA
ggggcaucguuucagaauunn

SEQ ID NO: 5 - RNA, 21 nt, Artificial Sequence
  siRNA
caucgacuuggucaaagugnn

SEQ ID NO: 6 - RNA, 21 nt, Artificial Sequence
  siRNA
cacuuugaccaagucgaugnn

SEQ ID NO: 7 - RNA, 21 nt, Artificial Sequence
  siRNA
aagcugacgagugaacuugnn

SEQ ID NO: 8 - RNA, 21 nt, Artificial Sequence
  siRNA
caaguucacucgucagcuunn

SEQ ID NO: 9 - DNA, 20 bp, Artificial Sequence
  antisense oligonucleotide
agctcctcgaccaatcacct

SEQ ID NO: 10 - DNA, 20 bp, Artificial Sequence
  antisense oligonucleotide
ggggcatcgtttcagaattt

SEQ ID NO: 11 - DNA, 20 bp, Artificial Sequence
  antisense oligonucleotide
cactttgaccaagtcgatgt

SEQ ID NO: 12 - DNA, 20 bp, Artificial Sequence
  antisense oligonucleotide
caagttcactcgtcagcttt

SEQ ID NO: 13 - RNA, 21 nt, Artificial Sequence
  siRNA
agccaagaggaaagaugggnn

SEQ ID NO: 14 - RNA, 21 nt, Artificial Sequence
  siRNA
cccaucuuuccucuuggcunn

SEQ ID NO: 15 - RNA, 21 nt, Artificial Sequence
  siRNA
gcgaauuaccucagaacagnn

SEQ ID NO: 16 - RNA, 21 nt, Artificial Sequence
  siRNA
cuguucugagguaauucgcnn

SEQ ID NO: 17 - RNA, 21 nt, Artificial Sequence
  siRNA
agguguuucugucucaugcnn

SEQ ID NO: 18 - RNA, 21 nt, Artificial Sequence
  siRNA
gcaugagacagaaacaccunn

SEQ ID NO: 19 - RNA, 21 nt, Artificial Sequence
  siRNA
uagaaggaacugggaucucnn

SEQ ID NO: 20 - RNA, 21 nt, Artificial Sequence
  siRNA
gagaucccaguuccuucuann

SEQ ID NO: 21 - RNA, 21 nt, Artificial Sequence
  siRNA
aacaaggguuccuccaguunn

SEQ ID NO: 22 - RNA, 21 nt, Artificial Sequence
  siRNA
aacuggaggaacccuuguunn

SEQ ID NO: 23 - RNA, 21 nt, Artificial Sequence
  siRNA
agucucgcaucagcuauagnn

SEQ ID NO: 24 - RNA, 21 nt, Artificial Sequence
  siRNA
cuauagcugaugcgagacunn

SEQ ID NO: 25 - RNA, 21 nt, Artificial Sequence
  siRNA
guuacuugaacgagaggugnn

SEQ ID NO: 26 - RNA, 21 nt, Artificial Sequence
  siRNA
caccucucguucaaguaacnn

SEQ ID NO: 27 - RNA, 21 nt, Artificial Sequence
  siRNA
cgagaggugaacauucugann

SEQ ID NO: 28 - RNA, 21 nt, Artificial Sequence
  siRNA
ucagaauguucaccucucgnn

SEQ ID NO: 29 - RNA, 21 nt, Artificial Sequence
  siRNA
aacauccuucagcuggugann

SEQ ID NO: 30 - RNA, 21 nt, Artificial Sequence
  siRNA
ucaccagcugaaggauguunn

SEQ ID NO: 31 - RNA, 21 nt, Artificial Sequence
  siRNA
ggcgaucuuauugaaguggnn

SEQ ID NO: 32 - RNA, 21 nt, Artificial Sequence
  siRNA
ccacuucaauaagaucgccnn

SEQ ID NO: 33 - RNA, 21 nt, Artificial Sequence
  siRNA
gaagcaaugguccaagaugnn

SEQ ID NO: 34 - RNA, 21 nt, Artificial Sequence
  siRNA
caucuuggaccauugcuucnn

SEQ ID NO: 35 - RNA, 21 nt, Artificial Sequence
  siRNA
auacccaacaauugcagcgnn

SEQ ID NO: 36 - RNA, 21 nt, Artificial Sequence
  siRNA
cgcugcaauuguuggguaunn

SEQ ID NO: 37 - RNA, 21 nt, Artificial Sequence
  siRNA
cagaucgaacacacccugann

SEQ ID NO: 38 - RNA, 21 nt, Artificial Sequence
  siRNA
ucaggguguguucgaucugnn

SEQ ID NO: 39 - RNA, 21 nt, Artificial Sequence
  siRNA
gaagggcagcgagcaggagnn

SEQ ID NO: 40 - RNA, 21 nt, Artificial Sequence
  siRNA
cuccugcucgcugcccuucnn

SEQ ID NO: 41 - RNA, 21 nt, Artificial Sequence
  siRNA
gggcagcgagcaggagagcnn

SEQ ID NO: 42 - RNA, 21 nt, Artificial Sequence
  siRNA
gcucuccugcucgcugcccnn

SEQ ID NO: 43 - RNA, 21 nt, Artificial Sequence
  siRNA
ccuuccuuucggaguaaucnn

SEQ ID NO: 44 - RNA, 21 nt, Artificial Sequence
  siRNA
gauuacuccgaaaggaaggnn

SEQ ID NO: 45 - RNA, 21 nt, Artificial Sequence
  siRNA
cgauacauggccccugaagnn

SEQ ID NO: 46 - RNA, 21 nt, Artificial Sequence
  siRNA
gacgugaagaucuaacugcnn

SEQ ID NO: 47 - RNA, 21 nt, Artificial Sequence
  siRNA
gaugaugcgagaguguuggnn

SEQ ID NO: 48 - RNA, 21 nt, Artificial Sequence
  siRNA
cugcucccucucuccacacnn

SEQ ID NO: 49 - RNA, 21 nt, Artificial Sequence
  siRNA
cuucaggggccauguaucgnn

SEQ ID NO: 50 - RNA, 21 nt, Artificial Sequence
  siRNA
gcaguuagaucuucacgucnn

SEQ ID NO: 51 - RNA, 21 nt, Artificial Sequence
  siRNA
ccaacacucucgcaucaucnn

SEQ ID NO: 52 - RNA, 21 nt, Artificial Sequence
  siRNA
guguggagagagggagcagnn

SEQ ID NO: 53 - RNA, 21 nt, Artificial Sequence
  siRNA
cgccaaggacaagaaccugnn

SEQ ID NO: 54 - RNA, 21 nt, Artificial Sequence
  siRNA
cagguucuuguccuuggcgnn

SEQ ID NO: 55 - RNA, 21 nt, Artificial Sequence
  siRNA
ugagaaccugaagaagucgnn

SEQ ID NO: 56 - RNA, 21 nt, Artificial Sequence
  siRNA
cgacuucuucagguucucann

SEQ ID NO: 57 - RNA, 21 nt, Artificial Sequence
  siRNA
gaagaacuccaagaaggugnn

SEQ ID NO: 58 - RNA, 21 nt, Artificial Sequence
  siRNA
caccuucuuggaguucuucnn

SEQ ID NO: 59 - RNA, 21 nt, Artificial Sequence
  siRNA
cagcagcuaccagaacaacnn

SEQ ID NO: 60 - RNA, 21 nt, Artificial Sequence
  siRNA
guuguucugguagcugcugnn

SEQ ID NO: 61 - RNA, 21 nt, Artificial Sequence
  siRNA
gcgaaggaccucauccagann

SEQ ID NO: 62 - RNA, 21 nt, Artificial Sequence
  siRNA
ucuggaugagguccuucgcnn

SEQ ID NO: 63 - RNA, 21 nt, Artificial Sequence
  siRNA
gcuuacgagaggaggauucnn

SEQ ID NO: 64 - RNA, 21 nt, Artificial Sequence
  siRNA
gaauccuccucucguaagcnn

SEQ ID NO: 65 - RNA, 21 nt, Artificial Sequence
  siRNA
cucaaagaugcccaucagcnn

SEQ ID NO: 66 - RNA, 21 nt, Artificial Sequence
  siRNA
gcugaugggcaucuuugagnn

SEQ ID NO: 67 - RNA, 21 nt, Artificial Sequence
  siRNA
cuucgacguggaugacgacnn

SEQ ID NO: 68 - RNA, 21 nt, Artificial Sequence
  siRNA
gucgucauccacgucgaagnn

SEQ ID NO: 69 - RNA, 21 nt, Artificial Sequence
  siRNA
ggcucaugagaggcuagaann

SEQ ID NO: 70 - RNA, 21 nt, Artificial Sequence
  siRNA
uucuagccucucaugagccnn

SEQ ID NO: 71 - RNA, 21 nt, Artificial Sequence
  siRNA
guuugugucacgaucugagnn

SEQ ID NO: 72 - RNA, 21 nt, Artificial Sequence
  siRNA
cucagaucgugacacaaacnn

SEQ ID NO: 73 - RNA, 21 nt, Artificial Sequence
  siRNA
gaugaaaaagauggccaggnn

SEQ ID NO: 74 - RNA, 21 nt, Artificial Sequence
  siRNA
ccuggccaucuuuuucaucnn

SEQ ID NO: 75 - RNA, 21 nt, Artificial Sequence
  siRNA
auguggcagaauugguuggnn

SEQ ID NO: 76 - RNA, 21 nt, Artificial Sequence
  siRNA
ccaaccaauucugccacaunn

SEQ ID NO: 77 - DNA, 20 bp, Artificial Sequence
  antisense oligonucleotide
cccatctttcctcttggctt

SEQ ID NO: 78 - DNA, 20 bp, Artificial Sequence
  antisense oligonucleotide
ctgttctgaggtaattcgct

SEQ ID NO: 79 - DNA, 20 bp, Artificial Sequence
  antisense oligonucleotide
gcatgagacagaaacacctt

SEQ ID NO: 80 - DNA, 20 bp, Artificial Sequence
  antisense oligonucleotide
gagatcccagttccttctat

SEQ ID NO: 81 - DNA, 20 bp, Artificial Sequence
  antisense oligonucleotide
aactggaggaacccttgttt

SEQ ID NO: 82 - DNA, 20 bp, Artificial Sequence
  antisense oligonucleotide
ctatagctgatgcgagactt

SEQ ID NO: 83 - DNA, 20 bp, Artificial Sequence
  antisense oligonucleotide
cacctctcgttcaagtaact

SEQ ID NO: 84 - DNA, 20 bp, Artificial Sequence
  antisense oligonucleotide
tcagaatgttcacctctcgt

SEQ ID NO: 85 - DNA, 20 bp, Artificial Sequence
  antisense oligonucleotide
tcaccagctgaaggatgttt

SEQ ID NO: 86 - DNA, 20 bp, Artificial Sequence
  antisense oligonucleotide
ccacttcaataagatcgcct

SEQ ID NO: 87 - DNA, 20 bp, Artificial Sequence
  antisense oligonucleotide
catcttggaccattgcttct

SEQ ID NO: 88 - DNA, 20 bp, Artificial Sequence
  antisense oligonucleotide
cgctgcaattgttgggtatt

SEQ ID NO: 89 - DNA, 19 bp, Artificial Sequence
  antisense oligonucleotide
tcagggtgtgttcgatctg

SEQ ID NO: 90 - DNA, 19 bp, Artificial Sequence
  antisense oligonucleotide
ctcctgctcgctgcccttc

SEQ ID NO: 91 - DNA, 19 bp, Artificial Sequence
  antisense oligonucleotide
gctctcctgctcgctgccc

SEQ ID NO: 92 - DNA, 19 bp, Artificial Sequence
  antisense oligonucleotide
gattactccgaaaggaagg

SEQ ID NO: 93 - DNA, 19 bp, Artificial Sequence
  antisense oligonucleotide
cttcaggggccatgtatcg

SEQ ID NO: 94 - DNA, 19 bp, Artificial Sequence
  antisense oligonucleotide
gcagttagatcttcacgtc

SEQ ID NO: 95 - DNA, 19 bp, Artificial Sequence
  antisense oligonucleotide
ccaacactctcgcatcatc

SEQ ID NO: 96 - DNA, 19 bp, Artificial Sequence
  antisense oligonucleotide
gtgtggagagagggagcag

SEQ ID NO: 97 - DNA, 19 bp, Artificial Sequence
  antisense oligonucleotide
caggttcttgtccttggcg

SEQ ID NO: 98 - DNA, 19 bp, Artificial Sequence
  antisense oligonucleotide
cgacttcttcaggttctca

SEQ ID NO: 99 - DNA, 19 bp, Artificial Sequence
  antisense oligonucleotide
caccttcttggagttcttc

SEQ ID NO: 100 - DNA, 19 bp, Artificial Sequence
  antisense oligonucleotide
gttgttctggtagctgctg

SEQ ID NO: 101 - DNA, 20 bp, Artificial Sequence
  antisense oligonucleotide
tctggatgaggtccttcgct

SEQ ID NO: 102 - DNA, 20 bp, Artificial Sequence
  antisense oligonucleotide
gaatcctcctctcgtaagct

SEQ ID NO: 103 - DNA, 20 bp, Artificial Sequence
  antisense oligonucleotide
gctgatgggcatctttgagt

SEQ ID NO: 104 - DNA, 20 bp, Artificial Sequence
  antisense oligonucleotide
gtcgtcatccacgtcgaagt

SEQ ID NO: 105 - DNA, 20 bp, Artificial Sequence
  antisense oligonucleotide
ttctagcctctcatgagcct

SEQ ID NO: 106 - DNA, 20 bp, Artificial Sequence
  antisense oligonucleotide
ctcagatcgtgacacaaact

SEQ ID NO: 107 - DNA, 20 bp, Artificial Sequence
  antisense oligonucleotide
cctggccatctttttcatct

SEQ ID NO: 108 - DNA, 20 bp, Artificial Sequence
  antisense oligonucleotide
ccaaccaattctgccacatt

SEQ ID NO: 109 - DNA, 20 bp, Artificial Sequence
  antisense oligonucleotide
agctcctcgaccaatcacct

SEQ ID NO: 110 - DNA, 20 bp, Artificial Sequence
  antisense oligonucleotide
ggggcatcgtttcagaattt

SEQ ID NO: 111 - DNA, 20 bp, Artificial Sequence
  antisense oligonucleotide
cactttgaccaagtcgatgt

SEQ ID NO: 112 - DNA, 20 bp, Artificial Sequence
  antisense oligonucleotide
caagttcactcgtcagcttt

SEQ ID NO: 113 - DNA, 1772 bp, Homo sapiens
gggctccggcctcagaggctgtgacaatggactatgactttaaagtgaagctgagcagcgagcgggagcgggtcgaggacctgtttgaatacgagggctgcaaagttggccgaggcacttatggtcacgtctacaaagccaagaggaaagatgggaaggatgataaagactatgctttaaaacaaatagaaggaactgggatctctatgtcggcatgtagagaaatagcattacttcgagagcttaagcatccaaacgtcatttctcttcaaaaggtgtttctgtctcatgctgataggaaggtgtggcttctgtttgactatgctgaacatgacctctggcatataatcaagtttcacagagcttctaaagcaaacaagaagccagttcagttacctcggggaatggtgaagtcactattatatcagatcctagatggtattcactacctgcatgctaactgggtgttgcacagagatttgaaacctgctaatattttagttatgggtgaaggtcctgagcgaggaagagtaaaaattgctgacatgggctttgcccgattatttaattcacctttgaagcctttagcagatttggatccagtggttgttacattctggtaccgagcccctgaactacttcttggagcaaggcattataccaaagctattgatatttgggctatagggtgtatatttgcagaactactaacgtcagaaccaatatttcactgtcgacaagaggacatcaaaactagtaatccttatcaccatgaccagctggacagaatattcaatgtaatgggatttcctgcagataaagattgggaagatataaaaaagatgcctgaacattcaacattaatgaaagatttcagaagaaatacgtataccaactgcagccttatcaagtatatggaaaaacataaagttaaaccagatagtaaagcattccacttgcttcagaagctgcttaccatggacccaataaagcgaattacctcagaacaggctatgcaggacccctatttcttagaagacccacttcctacatcagacgtttttgccggttgtcaaatcccttacccaaaacgagaatttttaacggaagaagaacctgatgacaaaggagacaaaaagaaccagcagcagcagcagggcaataaccacactaatggaactggccacccagggaatcaagacagcagtcacacacagggacccccgttgaagaaagtgagagttgttcctcctaccactacctcaggtggacttatcatgacctcagactatcagcgttccaatccacatgctgcctatcccaaccctggaccaagcacatcacagccgcagagcagcatgggatactcagctacctcccagcagcctccacagtactcacatcagacacatcggtactgagctgcatcggaatcttgtccatgcactgttgcgaatgctgcagggctgactgtgcagctctctgcgggaacctggtatgggccatgagaatgtactgtacaaccacatcttcaaaatgtccagtagccaagttccaccacttttcacagattggggtagtggcttccaagttgtacctattttggagttagacttgaaaagaaagtgctagcacagtttgtgttgtggatttgctacttccatagtttacttgacatggttcagactgaccaatgcatttttttcagtgacagtctgtagcagttgaagctgtgaatgtgctaggggcaagcatttgtctttgtatgtggt

SEQ ID NO: 114 - DNA, 3064 bp, Homo sapiens
atgtactcccaattacttctggaagtttctcaaagtactcctttatatatactgcagagtgtatttttcttcctcctcaactgagatctttccaacttgccaccatgcagctgccaatggtcctagttaagtaaaatgctgccatacctattttagactcagggaaaaatagcacccactcatttttatttttgctcaatataaaaatgaggatacttatgaggatacttaaacttttaggattagctagttttctaaaaatcgaattattcactcctttgtaaagtatgtaataggaatttgctctaataatcaatagattaaggtttaaaatttgaaaccatagtaatgtatgtttaacaccaatattttaagcctttttaaaaaccacaacccacattaagaaatacatttcatactgtgatcaagtacacacgcacacacacactctatacatatatgtctgtccaattaaaagtttcacagaaatttccaaggaggtatgctaaatattatctctttgattctactttatttttaaaaagtggtatcaacccacaaaatggatttcataacccactacgcagtttgataagatgctgttttagaccatgcttttcaccagttttgtggtcctattttgtccttttcatgtctatacaggatgcttctagtgctagttgctagcttttctctgatttccaggatggtaataggttaagaatttctctaaatggttatttcttttctttctgcagctctcacgtgtgaatatgtgtctagtgcatccttaacctgaggacttcaccagttcgaaattacagttttcaccatcaactaccttatcctttttggcctggttttcttcctcaaacagtggaaacatttttaaagttgcttttgttgcagagttaaacaaatggctgatagtggcttagataaaaaatccacaaaatgccccgactgttcatctgcttctcagaaagatgtactttgtgtatgttccagcaaaacaagggttcctccagttttggtggtggaaatgtcacagacatcaagcattggtagtgcagaatctttaatttcactggagagaaaaaaagaaaaaaatatcaacagagatataacctccaggaaagatttgccctcaagaacctcaaatgtagagagaaaagcatctcagcaacaatggggtcggggcaactttacagaaggaaaagttcctcacataaggattgagaatggagctgctattgaggaaatctatacctttggaagaatattgggaaaagggagctttggaatagtcattgaagctacagacaaggaaacagaaacgaagtgggcaattaaaaaagtgaacaaagaaaaggctggaagctctgctgtgaagttacttgaacgagaggtgaacattctgaaaagtgtaaaacatgaacacatcatacatctggaacaagtatttgaaacgccaaagaaaatgtaccttgtgatggagctttgtgaggatggagaactcaaagaaattctggataggaaagggcatttctcagagaatgagacaaggtggatcattcaaagtctcgcatcagctatagcatatcttcacaataatgatattgtacatagagatctgaaactggaaaatataatggttaaaagcagtcttattgatgataacaatgaaataaacttaaacataaaggtgactgattttggcttagcggtgaagaagcaaagtaggagtgaagccatgctgcaggccacatgtgggactcctatctatatggcccctgaagttatcagtgcccacgactatagccagcagtgtgacatttggagcataggcgtcgtaatgtacatgttattacgtggagaaccaccctttttggcaagctcagaagagaagctttttgagttaataagaaaaggagaactacattttgaaaatgcagtctggaattccataagtgactgtgctaaaagtgttttgaaacaacttatgaaagtagatcctgctcacagaatcacagctaaggaactactagataaccagtggttaacaggcaataaactttcttcggtgagaccaaccaatgtattagagatgatgaaggaatggaaaaataacccagaaagtgttgaggaaaacacaacagaagagaagaataagccgtccactgaagaaaagttgaaaagttaccaaccctggggaaatgtccctgatgccaattacacttcagatgaagaggaggaaaaacagtctactgcttatgaaaagcaatttcctgcaaccagtaaggacaactttgatatgtgcagttcaagtttcacatctagcaaactccttccagctgaaatcaagggagaaatggagaaaacccctgtgactccaagccaaggaacagcaaccaagtaccctgctaaatccggcgccctgtccagaaccaaaaagaaactctaaggttccctccagtgttggacagtacaaaaacaaagctgctcttgttagcactttgatgagggggtaggaggggaagaagacagccctatgctgagcttgtagccttttagctccacagagccccgccatgtgtttgcaccagcttaaaattgaagctgcttatctccaaagcagcataagctgcacatggcattaaaggacagccaccagtaggcttggcagtgggctgcagtggaaatcaactcaagatgtacacgaaggttttttaggggggcagataccttcaatttaaggctgtgggcacacttgctcatttttacttcaaattcttatgtttaggcacagctatttataggggaaaacaagaggccaaatatagtaatggaggtgccaaataattatgtgcactttgcactagaagactttgttagaaaattactaataaacttgccatacgtattacagcagaagtgcttcagtcattcacatgtgttcgtgagattttaggttgctatagattgtttaagacagcttattttaaatgtagaaaaataggagattttgtaactgcttgccattaacttgctgctaaattcccaa

SEQ ID NO: 115 - DNA, 3742 bp, Homo sapiens
gaattccttctctcctcctcctcgcccttctcctcgccctcctcctcctcctcgccctcccctcccgatcctcatccccttgccctcccccagcccagggacttttccggaaagtttttattttccgtctgggctctcggagaaagaagctcctggctcagcggctgcaaaactttcctgctgccgcgccgccagcccccgccctccgctgcccggccctgcgccccgccgagcgatgagcgcccctccggtcctgcggccgcccagtccgctgctgcccgtggcggcggcagctgccgcagcggccgccgcactggtcccagggtccgggcccgggcccgcgccgttcttggctcctgtcgcggccccggtcgggggcatctcgttccatctgcagatcggcctgagccgtgagccggtgctgctgctgcaggactcgtccggggactacagcctggcgcacgtccgcgagatggcttgctccattgtcgaccagaagttccctgaatgtggtttctacggaatgtatgataagatcctgctttttcgccatgaccctacctctgaaaacatccttcagctggtgaaagcggccagtgatatccaggaaggcgatcttattgaagtggtcttgtcacgttccgccacctttgaagactttcagattcgtccccacgctctctttgttcattcatacagagctccagctttctgtgatcactgtggagaaatgctgtgggggctggtacgtcaaggtcttaaatgtgaagggtgtggtctgaattaccataagagatgtgcatttaaaatacccaacaattgcagcggtgtgaggcggagaaggctctcaaacgtttccctcactggggtcagcaccatccgcacatcatctgctgaactctctacaagtgcccctgatgagccccttctgcaaaaatcaccatcagagtcgtttattggtcgagagaagaggtcaaattctcaatcatacattggacgaccaattcaccttgacaagattttgatgtctaaagttaaagtgccgcacacatttgtcatccactcctacacccggcccacagtgtgccagtactgcaagaagcttctgaaggggcttttcaggcagggcttgcagtgcaaagattgcagattcaactgccataaacgttgtgcaccgaaagtaccaaacaactgccttggcgaagtgaccattaatggagatttgcttagccctggggcagagtctgatgtggtcatggaagaagggagtgatgacaatgatagtgaaaggaacagtgggctcatggatgatatggaagaagcaatggtccaagatgcagagatggcaatggcagagtgccagaacgacagtggcgagatgcaagatccagacccagaccacgaggacgccaacagaaccatcagtccatcaacaagcaacaatatcccactcatgagggtagtgcagtctgtcaaacacacgaagaggaaaagcagcacagtcatgaaagaaggatggatggtccactacaccagcaaggacacgctgcggaaacggcactattggagattggatagcaaatgtattaccctctttcagaatgacacaggaagcaggtactacaaggaaattcctttatctgaaattttgtctctggaaccagtaaaaacttcagctttaattcctaatggggccaatcctcattgtttcgaaatcactacggcaaatgtagtgtattatgtgggagaaaatgtggtcaatccttccagcccatcaccaaataacagtgttctcaccagtggcgttggtgcagatgtggccaggatgtgggagatagccatccagcatgcccttatgcccgtcattcccaagggctcctccgtgggtacaggaaccaacttgcacagagatatctctgtgagtatttcagtatcaaattgccagattcaagaaaatgtggacatcagcacagtatatcagatttttcctgatgaagtactgggttctggacagtttggaattgtttatggaggaaaacatcgtaaaacaggaagagatgtagctattaaaatcattgacaaattacgatttccaacaaaacaagaaagccagcttcgtaatgaggttgcaattctacagaaccttcatcaccctggtgttgtaaatttggagtgtatgtttgagacgcctgaaagagtgtttgttgttatggaaaaactccatggagacatgctggaaatgatcttgtcaagtgaaaagggcaggttgccagagcacataacgaagtttttaattactcagatactcgtggctttgcggcaccttcattttaaaaatatcgttcactgtgacctcaaaccagaaaatgtgttgctagcctcagctgatccttttcctcaggtgaaactttgtgattttggttttgcccggatcattggagagaagtctttccggaggtcagtggtgggtacccccgcttacctggctcctgaggtcctaaggaacaagggctacaatcgctctctagacatgtggtctgttggggtcatcatctatgtaagcctaagcggcacattcccatttaatgaagatgaagacatacacgaccaaattcagaatgcagctttcatgtatccaccaaatccctggaaggaaatatctcatgaagccattgatcttatcaacaatttgctgcaagtaaaaatgagaaagcgctacagtgtggataagaccttgagccacccttggctacaggactatcagacctggttagatttgcgagagctggaatgcaaaatcggggagcgctacatcacccatgaaagtgatgacctgaggtgggagaagtatgcaggcgagcagcggctgcagtaccccacacacctgatcaatccaagtgctagccacagtgacactcctgagactgaagaaacagaaatgaaagccctcggtgagcgtgtcagcatcctctgagttccatctcctataatctgtcaaaacactgtggaactaataaatacatacggtcaggtttaacatttgccttgcagaactgccattattttctgtcagatgagaacaaagctgttaaactgttagcactgttgatgtatctgagttgccaagacaaatcaacagaagcatttgtattttgtgtgaccaactgtgttgtattaacaaaagttccctgaaacacgaaacttgttattgtgaatgattcatgttatatttaatgcattaaacctgtctccactgtgcctttgcaaatcagtgtttttcttactggagcttcattttggtaagagacagaatgtatctgtgaagtagttctgtttggtgtgtcccattggtgttgtcattgtaaacaaactcttgaagagtcgattatttccagtgttctatgaacaactccaaaacccatgtgggaaaaaaatgaatgaggagggtagggaataaaatcctaagacacaaatgcatgaacaagttttaatgtatagttttgaatcctttgcctgcctggtgtgcctcagtatatttaaactcaagacaatgcacctagctgtgcaagacctagtgctcttaagcctaaatgccttagaaatgtaaactgccatatataacagatacatttccctctttcttataatactctgttgtactatggaaaatcagctgctcagcaacctttcacctttgtgtatttttcaataataaaaaatattcttgtcaaaaaaaaaaaaa

SEQ ID NO: 116 - DNA, 2549 bp, Homo sapiens
  misc_feature (6)...(6): N is a, t, g, c, unknown, or other
cagtgngctccgggccgccggccgcagccagcacccgccgcgccgcagctccgggaccggccccggccgccgccgccgcgatgggcaacgccgccgccgccaagaagggcagcgagcaggagagcgtgaaagaattcttagccaaagccaaagaagattttcttaaaaaatgggaaagtcccgctcagaacacagcccacttggatcagtttgaacgaatcaagaccctcggcacgggctccttcgggcgggtgatgctggtgaaacacaaggagaccgggaaccactatgccatgaagatcctcgacaaacagaaggtggtgaaactgaaacagatcgaacacaccctgaatgaaaagcgcatcctgcaagctgtcaactttccgttcctcgtcaaactcgagttctccttcaaggacaactcaaacttatacatggtcatggagtacgtgcccggcggggagatgttctcacacctacggcggatcggaaggttcagtgagccccatgcccgtttctacgcggcccagatcgtcctgacctttgagtatctgcactcgctggatctcatctacagggacctgaagccggagaatctgctcattgaccagcagggctacattcaggtgacagacttcggtttcgccaagcgcgtgaagggccgcacttggaccttgtgcggcacccctgagtacctggcccctgagattatcctgagcaaaggctacaacaaggccgtggactggtgggccctgggggttcttatctatgaaatggccgctggctacccgcccttcttcgcagaccagcccatccagatctatgagaagatcgtctctgggaaggtgcgcttcccttcccacttcagctctgacttgaaggacctgctgcggaacctcctgcaggtagatctcaccaagcgctttgggaacctcaagaatggggtcaacgatatcaagaaccacaagtggtttgccacaactgactggattgccatctaccagaggaaggtggaagctcccttcataccaaagtttaaaggccctggggatacgagtaactttgacgactatgaggaagaagaaatccgggtctccatcaatgagaagtgtggcaaggagttttctgagttttaggggcatgcctgtgcccccatgggttttcttttttcttttttcttttttttggtcgggggggtgggagggttggattgaacagccagagggccccagagttccttgcatctaatttcacccccaccccaccctccagggttagggggagcaggaagcccagataatcagagggacagaaacaccagctgctccccctcatccccttcaccctcctgccccctctcccacttttcccttcctctttccccacagccccccagcccctcagccctcccagcccacttctgcctgttttaaacgagtttctcaactccagtcagaccaggtcttgctggtgtatccagggacagggtatggaaagaggggctcacgcttaactccagcccccacccacacccccatcccacccaaccacaggccccacttgctaagggcaaatgaacgaagcgccaaccttcctttcggagtaatcctgcctgggaaggagagatttttagtgacatgttcagtgggttgcttgctagaatttttttaaaaaaacaacaatttaaaatcttatttaagttccaccagtgcctccctccctccttcctctactcccacccctcccatgtccccccattcctcaaatccattttaaagagaagcagactgactttggaaagggaggcgctggggtttgaacctccccgctgctaatctcccctgggcccctccccggggaatcctctctgccaatcctgcgagggtctaggcccctttaggaagcctccgctctctttttccccaacagacctgtcttcacccttgggctttgaaagccagacaaagcagctgcccctctccctgccaaagaggagtcatcccccaaaaagacagagggggagccccaagcccaagtctttcctcccagcagcgtttccccccaactccttaattttattctccgctagattttaacgtccagccttccctcagctgagtggggagggcatccctgcaaaagggaacagaagaggccaagtccccccaagccacggcccggggttcaaggctagagctgctggggaggggctgcctgttttactcacccaccagcttccgcctcccccatcctgggcgcccctcctccagcttagctgtcagctgtccatcacctctcccccactttctcatttgtgcttttttctctcgtaatagaaaagtggggagccgctggggagccaccccattcatccccgtatttccccctctcataacttctccccatcccaggaggagttctcaggcctggggtggggccccgggtgggtgcgggggcgattcaacctgtgtgctgcgaaggacgagacttcctcttgaacagtgtgctgttgtaaacatatttgaaaactattaccaataaagtttgtt

SEQ ID NO: 117 - DNA, 2372 bp, Homo sapiens
cgctgctgggctgcggcggcggcggcggcggtggttactatggcggagtcggccggagcctcctccttcttcccccttgttgtcctcctgctcgccggcagcggcgggtccgggccccggggggtccaggctctgctgtgtgcgtgcaccagctgcctccaggccaactacacgtgtgagacagatggggcctgcatggtttccattttcaatctggatgggatggagcaccatgtgcgcacctgcatccccaaagtggagctggtccctgccgggaagcccttctactgcctgagctcggaggacctgcgcaacacccactgctgctacactgactactgcaacaggatcgacttgagggtgcccagtggtcacctcaaggagcctgagcacccgtccatgtggggcccggtggagctggtaggcatcatcgccggcccggtgttcctcctgttcctcatcatcatcattgttttccttgtcattaactatcatcagcgtgtctatcacaaccgccagagactggacatggaagatccctcatgtgagatgtgtctctccaaagacaagacgctccaggatcttgtctacgatctctccacctcagggtctggctcagggttacccctctttgtccagcgcacagtggcccgaaccatcgttttacaagagattattggcaagggtcggtttggggaagtatggcggggccgctggaggggtggtgatgtggctgtgaaaatattctcttctcgtgaagaacggtcttggttcagggaagcagagatataccagacggtcatgctgcgccatgaaaacatccttggatttattgctgctgacaataaagataatggcacctggacacagctgtggcttgtttctgactatcatgagcacgggtccctgtttgattatctgaaccggtacacagtgacaattgaggggatgattaagctggccttgtctgctgctagtgggctggcacacctgcacatggagatcgtgggcacccaagggaagcctggaattgctcatcgagacttaaagtcaaagaacattctggtgaagaaaaatggcatgtgtgccatagcagacctgggcctggctgtccgtcatgatgcagtcactgacaccattgacattgccccgaatcagagggtggggaccaaacgatacatggcccctgaagtacttgatgaaaccattaatatgaaacactttgactcctttaaatgtgctgatatttatgccctcgggcttgtatattgggagattgctcgaagatgcaattctggaggagtccatgaagaatatcagctgccatattacgacttagtgccctctgacccttccattgaggaaatgcgaaaggttgtatgtgatcagaagctgcgtcccaacatccccaactggtggcagagttatgaggcactgcgggtgatggggaagatgatgcgagagtgttggtatgccaacggcgcagcccgcctgacggccctgcgcatcaagaagaccctctcccagctcagcgtgcaggaagacgtgaagatctaactgctccctctctccacacggagctcctggcagcgagaactacgcacagctgccgcgttgagcgtacgatggaggcctacctctcgtttctgcccagccctctgtggccaggagccctggcccgcaagagggacagagcccgggagagactcgctcactcccatgttgggtttgagacagacaccttttctatttacctcctaatggcatggagactctgagagcgaattgtgtggagaactcagtgccacacctcgaactggttgtagtgggaagtcccgcgaaacccggtgcatctggcacgtggccaggagccatgacaggggcgcttgggaggggccggaggaaccgaggtgttgccagtgctaagctgccctgagggtttccttcggggaccagcccacagcacaccaaggtggcccggaagaaccagaagtgcagcccctctcacaggcagctctgagccgcgctttcccctcctccctgggatggacgctgccgggagactgccagtggagacggaatctgccgctttgtctgtccagccgtgtgtgcatgtgccgaggtgcgtcccccgttgtgcctggttcgtgccatgcccttacacgtgcgtgtgagtgtgtgtgtgtgtctgtaggtgcgcacttacctgcttgagctttctgtgcatgtgcaggtcgggggtgtggtcgtcatgctgtccgtgcttgctggtgcctcttttcagtagtgagcagcatctagtttccctggtgcccttccctggaggtctctccctcccccagagcccctcatgccacagtggtactctgtgt

SEQ ID NO: 118 - DNA, 1097 bp, Homo sapiens
aaactcagaattttcgcgggctcggtgagcggttttatccctccggccggcaggctgggcgcagggggcgagcccccgcccggcgcgcagcagcaccatgggcacggtgctgtccctgtctcccagctaccggaaggccacgctgtttgaggatggcgcggccaccgtgggccactatacggccgtacagaacagcaagaacgccaaggacaagaacctgaagcgccactccatcatctccgtgctgccttggaagagaatcgtggccgtgtcggccaagaagaagaactccaagaaggtgcagcctaacagcagctaccagaacaacatcacgcacctcaacaatgagaacctgaagaagtcgctgtcgtgcgccaacctgtccacattcgcccagcccccaccggcccagccgcctgcacccccggccagccagctctcgggttcccagaccgggggctcctcctcagtcaagaaagcccctcaccctgccgtcacctccgcagggacgcccaaacgggtcatcgtccaggcgtccaccagtgagctgcttcgctgcctgggtgagtttctctgccgccggtgctaccgcctgaagcacctgtcccccacggaccccgtgctctggctgcgcagcgtggaccgctcgctgcttctgcagggctggcaggaccagggcttcatcacgccggccaacgtggtcttcctctacatgctctgcagggatgttatctcctccgaggtgggctcggatcacgagctccaggccgtcctgctgacatgcctgtacctctcctactcctacatgggcaacgagatctcctacccgctcaagcccttcctggtggagagctgcaaggaggccttttgggaccgttgcctctctgtcatcaacctcatgagctcaaagatgctgcagataaatgccgacccacactacttcacacaggtcttctccgacctgaagaacgagagcggccaggaggacaagaagcggctcctcctaggcctggatcggtgagcactgtagcctgcgtcatggctcaaggattcaatgcatttttaagaatttattattaaatcagttttgtgtacag

SEQ ID NO: 119 - DNA, 6782 bp, Homo sapiens
gggcggggctgagggcggcgggggcgggccgcccgagctgggagggcggcggcgccgaggggaggagagcggcccatggacccgcggggcccggcgccccagactctgcgccgtcgggacggagcccaagatgtcggcctaggccggggcgcgacgacgcggacggggcggcgaggaggcgccgctgctgccggggctcgcagccgccgagcccccgagggcgcgccctgacggactggccgagccggcggtgagaggccggcgcgtcgggagcgggccgcgcggcaccatgtcggccaaggtgcggctcaagaagctggagcagctgctcctggacgggccctggcgcaacgagagcgccctgagcgtggaaacgctgctcgacgtgctcgtctgcctgtacaccgagtgcagccactcggccctgcgccgcgacaagtacgtggccgagttcctcgagtgggctaaaccatttacacagctggtgaaagaaatgcagcttcatcgagaagactttgaaataattaaagtaattggaagaggtgcttttggtgaggttgctgttgtcaaaatgaagaatactgaacgaatttatgcaatgaaaatcctcaacaagtgggagatgctgaaaagagcagagaccgcgtgcttccgagaggagcgcgatgtgctggtgaacggcgactgccagtggatcaccgcgctgcactacgcctttcaggacgagaaccacctgtacttagtcatggattactatgtgggtggtgatttactgaccctgctcagcaaatttgaagacaagcttccggaagatatggcgaggttctacattggtgaaatggtgctggccattgactccatccatcagcttcattacgtgcacagagacattaaacctgacaatgtccttttggacgtgaatggtcatatccgcctggctgactttggatcatgtttgaagatgaatgatgatggcactgtgcagtcctccgtggccgtgggcacacctgactacatctcgccggagatcctgcaggcgatggaggacggcatgggcaaatacgggcctgagtgtgactggtggtctctgggtgtctgcatgtatgagatgctctatggagaaacgccgttttatgcggagtcactcgtggagacctatgggaagatcatgaaccatgaagagcgattccagttcccatcccatgtcacggatgtatctgaagaagcgaaggacctcatccagagactgatctgcagtagagaacgccggctggggcagaatggaatagaggatttcaaaaagcatgcgttttttgaaggtctaaattgggaaaatatacgaaacctagaagcaccttatattcctgatgtgagcagtccctctgacacatccaacttcgacgtggatgacgacgtgctgagaaacacggaaatattacctcctggttctcacacaggcttttctggattacatttgccattcattggttttacattcacaacggaaagctgtttttctgatcgaggctctctgaagagcataatgcagtccaacacattaaccaaagatgaggatgtgcagcgggacctggagcacagcctgcagatggaagcttacgagaggaggattcggaggctggaacaggagaagctggagctgagcaggaagctgcaagagtccacccagaccgtgcagtccctccacggctcatctcgggccctcagcaattcaaaccgagataaagaaatcaaaaagctaaatgaagaaatcgaacgcttgaagaataaaatagcagattcaaacaggctcgagcgacagcttgaggacacagtggcgcttcgccaagagcgtgaggactccacgcagcggctgcgggggctggagaagcagcaccgcgtggtccggcaggagaaggaggagctgcacaagcaactggttgaagcctcagagcggttgaaatcccaggccaaggaactcaaagatgcccatcagcagcgaaagctggccctgcaggagttctcggagctgaacgagcgcatggcagagctccgtgcccagaagcagaaggtgtcccggcagctgcgagacaaggaggaggagatggaggtggccacgcagaaggtggacgccatgcggcaggaaatgcggagagctgagaagctcaggaaagagctggaagctcagcttgatgatgctgttgctgaggcctccaaggagcgcaagcttcgtgagcacagcgagaacttctgcaagcaaatggaaagcgagctggaggccctcaaggtgaagcaaggaggccggggagcgggtgccaccttagagcaccagcaagagatttccaaaatcaaatccgagctggagaagaaagtcttattttatgaagaggaattggtcagacgtgaggcctcccatgtgctagaagtgaaaaatgtgaagaaggaggtgcatgattcagaaagccaccagctggccctgcagaaagaaatcttgatgttaaaagataagttagaaaagtcaaagcgagaacggcataacgagatggaggaggcagtaggtacaataaaagataaatacgaacgagaaagagcgatgctgtttgatgaaaacaagaagctaactgctgaaaatgaaaagctctgttcctttgtggataaactcacagctcaaaatagacagctggaggatgagctgcaggatctggcagccaagaaggagtcagtggcccactgggaagctcagattgcggaaatcattcagtgggtcagtgacgagaaagatgcccggggttaccttcaagctcttgcttccaagatgaccgaagagctcgaggctttgaggagttctagtctggggtcaagaacactggacccgctgtggaaggtgcgccgcagccagaagctggacatgtccgcgcggctggagctgcagtcggccctggaggcggagatccgggccaagcagcttgtccaggaggagctcaggaaggtcaaggacgccaacctcaccttggaaagcaaactaaaggattccgaagccaaaaacagagaattattagaagaaatggaaattttgaagaaaaagatggaagaaaaattcagagcagatactgggctcaaacttccagattttcaggattccatttttgagtatttcaacactgctcctcttgcacatgacctgacatttagaaccagctcagctagtgagcaagaaacacaagctccgaagccagaagcgtccccgtcgatgtctgtggctgcatcagagcagcaggaggacatggctcggcccccgcagaggccatccgctgtgccgttgcccaccacgcaggccctggctctggctggaccgaagccaaaagctcaccagttcagcatcaagtccttctccagccctactcagtgcagccactgcacctccctgatggttgggctgatccggcagggctacgcctgcgaggtgtgttcctttgcttgccacgtgtcctgcaaagacggtgccccccaggtgtgcccaatacctcccgagcagtccaagaggcctctgggcgtggacgtgcagcgaggcatcggaacagcctacaaaggccatgtcaaggtcccaaagcccacgggggtgaagaagggatggcagcgcgcatatgcagtcgtctgtgagtgcaagctcttcctgtatgatctgcctgaaggaaaatccacccagcctggtgtcattgcgagccaagtcttggatctcagagatgacgagttttccgtgagctcagtcctggcctcagatgtcattcatgctacacgccgagatattccatgtatattcagggtgacggcctctctcttaggtgcaccttctaagaccagctcgctgctcattctgacagaaaatgagaatgaaaagaggaagtgggttgggattctagaaggactccagtccatccttcataaaaaccggctgaggaatcaggtcgtgcatgttcccttggaagcctacgacagctcgctgcctctcatcaaggccatcctgacagctgccatcgtggatgcagacaggattgcagtcggcctagaagaagggctctatgtcatagaggtcacccgagatgtgatcgtccgtgccgctgactgtaagaaggtacaccagatcgagcttgctcccagggagaagatcgtaatcctcctctgtggccggaaccaccatgtgcacctctatccgtggtcgtcccttgatggagcggaaggcagctttgacatcaagcttccggaaaccaaaggctgccagctcatggccacggccacactcaagaggaactctggcacctgcctgtttgtggccgtgaaacggctgatcctttgctatgagatccagagaacgaagccattccacagaaagttcaatgagattgtggctcccggcagcgtgcagtgcctggcggtgctcagggacaggctctgtgtgggctacccttctgggttctgcctgctgagcatccagggggacgggcagcctctaaacctggtaaatcccaatgacccctcgcttgcgttcctctcacaacagtcttttgatgccctttgtgctgtggagctcgaaagcgaggagtacctgctttgcttcagccacatgggactgtacgtggacccgcaaggccggagggcacgcgcgcaggagctcatgtggcctgcggctcctgtcgcctgtagttgcagccccacccacgtcacggtgtacagcgagtatggcgtggacgtctttgatgtgcgcaccatggagtgggtgcagaccatcggcctgcggaggataaggcccctgaactctgaaggcaccctcaacctcctcaactgcgagcctccacgcttgatctacttcaagagcaagttctcgggagcggttctcaacgtgccggacacctccgacaacagcaagaagcagatgctgcgcaccaggagcaaaaggcggttcgtcttcaaggtcccagaggaagagagactgcagcagaggcgagagatgcttagagacccagaattgagatccaaaatgatatccaacccaaccaacttcaaccacgtggcccacatgggcccaggcgacggcatgcaggtgctcatggacctgcctctgagtgctgtgcccccctcccaggaggaaaggccgggccccgctcccaccaacctggctcgccagcctccatccaggaacaagccctacatctcgtggccctcatcaggtggatcggagcctagcgtgactgtgcctctgagaagtatgtctgatccagaccaggactttgacaaagagcctgattcggactccaccaaacactcaactccatcgaatagctccaaccccagcggcccaccgagccccaactccccccacaggagccagctccccctcgaaggcctggagcagccggcctgtgacacctgaagccgccagctcgccacaggggccagggagctggagatggcctccagcgtcagtgccaagactgagcgggccctccagtgttgtccaaggaaatgtagaatcactttgtagatatggagatgaagaagacaaatctttattataatattgatcagttttatgccgcattgttcgtggcagtagaccacatctgttcgtctgcacagctgtgaggcgatgctgttccatctgcacatgaaggacccccatacagcctgtctcccacccctgacaacccgagagggcatatggggccctgccaacaccacttcctcagcagaaacccgtcatgacgcggctgcttcggaagcagacatctggggacacagcctcagtacccagtcttttccctagttcctgaaactttcctaggaccttaagagaatagtaggaggtcctatagcattcccagtgtcactagaattttgaagacaggaaagtggaggttagtctgtggcctttttttcatttagccattgcacagtcagctgcagaagtcctgctgaccacctagtcatggacaaaggcccaggaccagtgacaccctgcgtccctgtgtgcattaagttcattctgggtcgcagccatgaagtgtcaccagtatctactactgtgaagtcagctgtgctgttttccattcgcttccacggcttctgcctcctgccataaaaccagcgagtgtcgtggtgcaggcaggccctgtggcctgctgggctgagggaagtcagagccccagggcgccacgaagcagccactgggataccccaccccgccccgccctgcccccccccccccccaccagtcctgcccccgcatggagcccccgtgattagtagcccgtatgatcacgtagacccacccaacacactcctgcacactggccccggcccacggcacagcaatcccctgcgcgtggatttcacctcaccctttgtaccagatgttgagtgaccagctctgtggccctgtgtcgtcagaggcttgtgattaactgtggcggcagacacagcttgtccacagcttgggccaggcttcccctgtcctcccaccggtcggctgcttggcaaggctgttcaggacgtgcacttccccaagtcggcactgagtggcccagcaccgcctagccctgccaccccactgccctcctgggccttctgctggatgggcacctggggggttctggtttttacttttttaatgtaagtctcagtctttgtaattaattattgaattgtgagaacatttttgaacaatttacctgtcaataaagcagaagacggcagttttaaagttaaaaaaaaaaaaaaaaaaaaaaaaa

SEQ ID NO: 120 - DNA, 2201 bp, Homo sapiens
caactacgagccacgagtttgcagatggggctgctcggcggcgcctgtggctgagggagagcagcggcggcggggagcgaccgggagcggcggcagcggcggcgcggaggcggctgaggtgcgagccggactaaatcattttgctactttaaaaaaatcacgaaagtacattatttgaagtttggagaagaaagggatttggtaacaaaggacagccatttccattttaagcagctaaacagcaggagagatttctgtaagaaggtaccagctcagattccattgttcatcattttgcaatgcagcaagtcttggaaaaccttacggagctgccctcgtctactggagcagaagaaatagacctaattttcctcaagggaattatggagaatcctattgtaaaatcacttgctaaggctcatgagaggctagaagattccaaactagaagctgtcagtgacaataacttggaattagtcaatgaaattcttgaagacatcactcctctaataaatgtggatgaaaatgtggcagaattggttggtatactcaaagaacctcacttccagtcactgttggaggcccatgatattgtggcatcaaagtgttatgattcacctccatcaagcccagaaatgaataattcttctatcaataatcagttattaccagtagatgccattcgtattcttggtattcacaaaagagctggggaaccactgggtgtgacatttagggttgaaaataatgatctggtaattgcccgaatcctccatgggggaatgatagatcgacaaggtctacttcatgtgggagatataattaaagaagtcaatggccatgaggttggaaataatccaaaggaattacaagaattactgaaaaatattagtggaagtgtcaccctaaaaatcttaccaagttatagagataccattactcctcaacaggtatttgtgaagtgtcattttgattataatccatacaatgacaacctaataccttgcaaagaagcaggattgaagttttccaaaggagaaattcttcagattgtaaatagagaagatccaaattggtggcaggctagccatgtaaaagagggaggaagcgctggtctcattccaagccagttcctggaagagaagagaaaggcatttgttagaagagactgggacaattcaggacctttttgtggaactataagtagcaaaaaaaagaaaaagatgatgtatctcacaaccagaaatgcagaatttgatcgtcatgaaatccagatatatgaggaggtagccaaaatgcctcccttccagagaaaaacattagtattgataggagctcaaggtgtaggccgaagaagcttgaaaaacaggttcatagtattgaatcccactagatttggaactacggtgccatttacttcacggaaaccaagggaagatgaaaaagatggccaggcatataagtttgtgtcacgatctgagatggaagcagatattaaagctggaaagtatttggaacatggggaatatgaaggaaatctctatggaaccaaaattgattctattcttgaggttgtccaaactggacggacttgcattctggatgtcaacccacaagcactgaaagtattgaggacatcagagtttatgccctatgtggtatttattgcggctccggagctagagacgttacgtgccatgcacaaggctgtggtggatgcaggaatcactaccaagcttctgaccgactctgacttgaagaaaacagtggatgaaagtgcacggattcagagagcatacaaccactattttgatttgatcatcataaatgataatctagacaaagcctttgaaaaactgcaaactgccatagagaaactgagaatggaaccacagtgggtcccaatcagctgggtttactgatgattcagtaaggttaacaatgaaaattaaactcttaaaaagtgactgcaacaaataaaccttctactgagaaaatacatcacagatagaagattatctgctaagtccaggcatttttatggtgtagattgaaataatagtacacttctgaatttttatataaaatgtggttggaaggtgtactaatatataatttatcttaatttttctaactttgtatggataatctttctattcatatcacataaagaaatgcgttgaagcaaaaaaaaaaaaaaa

SEQ ID NO: 121 - DNA, 4917 bp, Homo sapiens
atgtctggagaagtgcgtttgaggcagttggagcagtttattttggacgggcccgctcagaccaatgggcagtgcttcagtgtggagacgttactggatatactcatctgcctttatgatgaatgcaataattctccattgagaagagagaagaacattctcgaatacctagaatgggctaaaccatttacttctaaagtgaaacaaatgcgattacatagagaagactttgaaatattaaaggtgattggtcgaggagcttttggggaggttgctgtagtaaaactaaaaaatgcagataaagtgtttgccatgaaaatattgaataaatgggaaatgctgaaaagagctgagacagcatgttttcgtgaagaaagggatgtattagtgaatggagacaataaatggattacaaccttgcactatgctttccaggatgacaataacttatacctggttatggattattatgttggtggggatttgcttactctactcagcaaatttgaagatagattgcctgaagatatggctagattttacttggctgagatggtgatagcaattgactcagttcatcagctacattatgtacacagagacattaaacctgacaatatactgatggatatgaatggacatattcggttagcagattttggttcttgtctgaagctgatggaagatggaacggttcagtcctcagtggctgtaggaactccagattatatctctcctgaaatccttcaagccatggaagatggaaaagggagatatggacctgaatgtgactggtggtctttgggggtctgtatgtatgaaatgctttacggagaaacaccattttatgcagaatcgctggtggagacatacggaaaaatcatgaaccacaaagagaggtttcagtttccagcccaagtgactgatgtgtctgaaaatgctaaggatcctattcgaaggctcatttgtggcagagaacatcgacttggtcaaagtggaatagaagactttaagaaacacccatttttcagtggaattgactgggataatattcggaactgtgaagcaccttatattccagaagttagtagcccaacagatacatcgaattttgatgtagatgatgattgtttaaaaaattctgaaacgatgcccccaccaacacatactgcattttctggccaccatctgccatttgttggttttacatatactagtagctgtgtactttctgatcggagctgtttaagagttacggctggtcccacctcactggatcttgatgttaatgttcagaggactctagacaacaacttagcaactgaagcttatgaaagaagaattaagcgccttgagcaagaaaaacttgaactcagtagaaaacttcaagagtcaacacagactgtccaagctctgcagtattcaactgttgatggtccactaacagcaagcaaagatttagaaataaaaaacttaaaagaagtaattgaaaaactaagaaaacaagtaacagaatcaagtcatttggaacagcaacttgaagaagctaatgctgtgaggcaagaactagatgatgcttttagacaaatcaaggcttatgaaaaacaaatcaaaacgttacaacaagaaagagaagatctaaataagctggaagttcatacagaagctctagctgctgaagcatctaaagacaggaagctacgtgaacagagtgagcactattctaagcaactggaaaatgaattggagggactgaagcaaaaacaaattagttactcaccaggagtatgcagcatagaacatcagcaagagataaccaaactaaagactgatttggaaaagaaaagtatcttttatgaagaagaattatctaaaagagaaggaatacatgcaaatgaaataaaaaatcttaagaaagaactgcatgattcagaaggtcagcaacttgctctcaacaaagaaattatgattttaaaagacaaattggaaaaaaccagaagagaaagtcaaagtgaaagggaggaatttgaaagtgagttcaaacaacaatatgaacgagaaaaagtgttgttaactgaagaaaataaaaagctgacgagtgaacttgataagcttactactttgtatgagaacttaagtatacacaaccagcagttagaagaagaggttaaagatctagcagacaagaaagaatcagttgcacattgggaagcccaaatcacagaaataattcagtgggtcagcgatgaaaaggatgcacgagggtatcttcaggccttagcttctaaaatgactgaagaattggaggcattaagaaattccagcttgggtacacgagcaacagatatgccctggaaaatgcgtcgttttgcgaaactggatatgtcagctagactggagttgcagtcggctctggatgcagaaataagagccaaacaggccatccaagaagagttgaataaagttaaagcatctaatatcataacagaatgtaaactaaaagattcagagaagaagaacttggaactactctcagaaatcgaacagctgataaaggacactgaagagcttagatctgaaaagggtatagagcaccaagactcacagcattctttcttggcatttttgaatacgcctaccgatgctctggatcaatttgaaactgtagactccactccactttcagttcacacaccaaccttaaggaaaaaaggatgtcctggttcaactggctttccacctaagcgcaagactcaccagttttttgtaaaatcttttactactcctaccaagtgtcatcagtgtacctccttgatggtgggtttaataagacagggctgttcatgtgaagtgtgtggattctcatgccatataacttgtgtaaacaaagctccaaccacttgtccagttcctcctgaacagacaaaaggtcccctgggtatagatcctcagaaaggaataggaacagcatatgaaggtcatgtcaggattcctaagccagctggagtgaagaaagggtggcagagagcactggctatagtgtgtgacttcaaactctttctgtacgatattgctgaaggaaaagcatctcagcccagtgttgtcattagtcaagtgattgacatgagggatgaagaattttctgtgagttcagtcttggcttctgatgttatccatgcaagtcggaaagatataccctgtatatttagggtcacagcttcccagctctcagcatctaataacaaatgttcaatcctgatgctagcagacactgagaatgagaagaataagtgggtgggagtgctgagtgaattgcacaagattttgaagaaaaacaaattcagagaccgctcagtctatgttcccaaagaggcttatgacagcactctacccctcattaaaacaacccaggcagccgcaatcatagatcatgaaagaattgctttgggaaacgaagaagggttatttgttgtacatgtcaccaaagatgaaattattagagttggtgacaataagaagattcatcagattgaactcattccaaatgatcagcttgttgctgtgatctcaggacgaaatcgtcatgtacgactttttcctatgtcagcattggatgggcgagagaccgatttttacaagctgtcagaaactaaagggtgtcaaaccgtaacttctggaaaggtgcgccatggagctctcacatgcctgtgtgtggctatgaaaaggcaggtcctctgttatgaactatttcagagcaagacccgtcacagaaaatttaaagaaattcaagtcccatataatgtccagtggatggcaatcttcagtgaacaactctgtgtgggattccagtcaggatttctaagataccccttgaatggagaaggaaatccatacagtatgctccattcaaatgaccatacactatcatttattgcacatcaaccaatggatgctatctgcgcagttgagatctccagtaaagaatatctgctgtgttttaacagcattgggatatacactgactgccagggccgaagatctagacaacaggaattgatgtggccagcaaatccttcctcttgttgttacaatgcaccatatctctcggtgtacagtgaaaatgcagttgatatctttgatgtgaactccatggaatggattcagactcttcctctcaaaaaggttcgacccttaaacaatgaaggatcattaaatcttttagggttggagaccattagattaatatatttcaaaaataagatggcagaaggggacgaactggtagtacctgaaacatcagataatagtcggaaacaaatggttagaaacattaacaataagcggcgttattccttcagagtcccagaagaggaaaggatgcagcagaggagggaaatgctacgagatccagaaatgagaaataaattaatttctaatccaactaattttaatcacatagcacacatgggtcctggagatggaatacagatcctgaaagatctgcccatgaaccctcggcctcaggaaagtcggacagtattcagtggctcagtcagtattccatctatcaccaaatcccgccctgagccaggccgctccatgagtgctagcagtggcttgtcagcaaggtcatccgcacagaatggcagcgcattaaagagggaattctctggaggaagctacagtgccaagcggcagcccatgccctccccgtcagagggctctttgtcctccggaggcatggaccaaggaagtgatgccccagcgagggactttgacggagaggactctgactctccgaggcattccacagcttccaacagttccaacctaagcagccccccaagcccagtttcaccccgaaaaaccaagagcctctccctggagagcactgaccgcgggagctgggacccgtga

(19) United States
(12) Patent Application Publication
(10) Pub. No.: US 2004/0180844 A1
(43) Pub. Date: Sep. 16, 2004

(54) Method of killing cancer cells

(76) Inventors: Stephen W. Fesik, Gurnee, IL (US); Donald N. Halbert, Libertyville, IL (US); Randy E. Metzger, Gurnee, IL (US); Jeffrey A. McDowell, Grayslake, IL (US); Mark E. Schurdak, Antioch, IL (US); Susan E. Morgan-Lappe, Chicago, IL (US); Aparna V. Sarthy, Waukegan, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/385,163

(22) Filed: Mar. 10, 2003

(51) Int. Cl. A61K 48/00

(52) U.S. Cl. 514/44

(57) ABSTRACT

A method of killing cancer cells comprising inhibiting the function of a gene selected from the group consisting of CDK8, STK33, PRKCM, PRKACA, ACVR1B, CDK5R1, CDC42BPB, MPP6, and CDC42BPA; pharmaceutical compositions comprising an inhibitor of the same, and a method of detecting cellular hyperplasia.

### TECHNICAL FIELD OF THE INVENTION

The present invention relates to methods of selectively killing cancer cells, detecting cancer cells, and compositions useful for killing cancer cells.

### BACKGROUND OF THE INVENTION

Many methods exist for killing or inhibiting the growth or propagation of hyperplastic, precancerous, and cancerous conditions in mammals. Unfortunately, these methods are still in need of improvement. For example, treatments could be improved by increasing their effectiveness, the duration or completeness of the therapeutic effect, the speed of their action, and other metrics of therapeutic performance.

Many therapeutic interventions improve the health status of mammals with hyperplastic, precancerous, and cancerous conditions. For example, one way of treating hyperplastic, precancerous, and cancerous conditions in mammals is to inhibit a cellular function critical for the progression of the condition or disease. Another way of treating hyperplastic, precancerous, and cancerous conditions in mammals is to inhibit a cellular function necessary for the survival of hyperplastic cells or dysplasias. Yet another way of treating hyperplastic, precancerous, and cancerous conditions in mammals is to render the cells more susceptible to bodily processes and/or other agents that control such conditions or diseases. The skilled artisan will appreciate that additional modes of therapy also exist and are well known in the art. New therapeutic methods could be developed, and existing therapeutic methods improved, if gene products could be identified that are important to the survival, or proliferation of hyperplasias and dysplasias. Thus, a need exists in the art for new therapeutic compositions and methods of applying or administering the same to treat hyperplastic, precancerous, and cancerous conditions in mammals in need of such treatment.

Signaling through the granulocyte-macrophage colony-stimulating factor receptor is mediated by 2 receptor subunits. The alpha subunit, which binds to GMCSF, has a short intracytoplasmic C-terminal tail that is essential for GMCSF-mediated growth stimulation. Zhao et al., J. Biol. Chem., 272, 10013-10020 (1997) used the intracytoplasmic domain of the alpha subunit of the GMCSF receptor to search for proteins that may be important for signal transduction by GMCSF. A serine/threonine protein kinase, PK428, was identified. PK428 is now known as CDC42BPA and can be accessed in the GenBank database at NM_{—}014826 (GI: 28274696).

This protein product of PK428 (part of CDC42BPA) is a 496-amino acid protein having an N-terminal kinase domain similar to the kinase domain of myotonic dystrophy protein kinase (“DMPK”). The PK428 gene product also contains a predicted helical region following the kinase domain, and a hydrophobic domain, both of which are similar to those found in DMPK. RNAs from human tissues contain a 10-kb mRNA in heart, brain, skeletal muscle, kidney, and pancreas, and 3.8- and 10-kb transcripts in a variety of human cell lines. Zhao et al. also found that PK428 is capable of autophosphorylation, as well as phosphorylation of histone H1 and a peptide substrate containing a cyclic AMP-dependent protein kinase phosphorylation site.

The PK428 gene resides at 1q41 -q42, a region thought to contain a gene associated with rippling muscle disease. Comparative genomic hybridization have shown that 1q41-q42 tends to be amplified in breast cancers and BRCA1 patients, although this region is massive and contains at least 20 gene sequences other than PK428. Additionally, the present inventors have found that the gene is not differentially expressed in lung, colon, and ovary tumor tissues compared to non-cancerous tissues of the same type.

CDK8 is a cyclin-dependent kinase. Cyclins are positive regulatory subunits of cyclin-dependent kinases (CDKs). Schultz et al., Cell Growth Differ., 4, 821-830 (1993) isolated cDNAs corresponding to the entire coding region of CDK8. The predicted 464-amino acid protein contains the sequence motifs and 11 sub-domains characteristic of a serine/threonine-specific kinase. CDK8 migrates as a 53-kD protein on Western blots of HeLa cell extracts. Co-immunoprecipitation experiments have revealed that CDK8 interacts with cyclin C both in vitro and in vivo. Tassan et al., Proc. Nat. Acad. Sci. (USA), 92, 8871-8875 (1995) has suggested that CDK8-cyclin C might be functionally associated with the mammalian transcription apparatus.

Mammalian CDK8 and cyclin C are components of the RNA polymerase II holoenzyme complex, where they function as a protein kinase that phosphorylates the C-terminal domain of the largest subunit of RNA polymerase II. The CDK8/cyclin C protein complex is also found in a number of mammalian ‘Mediator’-like protein complexes, which repress activated transcription independently of the C-terminal domain in vitro. Akoulitchev et al., Nature, 407, 102-106 (2000) disclosed that CDK8/cyclin C can regulate transcription. CDK8 phosphorylates mammalian cyclin H at serine-5 and serine-304 both in vitro and in vivo. This phosphorylation represses both the ability of TFIIH to activate transcription and its C-terminal kinase activity. In addition, mimicking CDK8 phosphorylation of cyclin H in vivo has a dominant-negative effect on cell growth. Akoulitchev et al. concluded that their results linked the Mediator-complex and the basal transcription machinery by a regulatory pathway involving 2 cyclin-dependent kinases. This pathway appears to be unique to higher organisms.

The CDK8 gene maps to 13q12.

STK33 encodes a novel serine/threonine protein kinase and was recently discovered to be located on human chromosome 11p15.3. STK33 is differentially expressed in normal and malignant tissues and studies suggests that it may belong to the calcium/calmodulin-dependent protein kinase family of proteins.

PRKCM encodes a cytosolic serine-threonine kinase that binds to the trans-Golgi network and regulates the fission of transport carriers specifically destined to the cell surface. The 912-amino acid PRKCM protein has a molecular mass of about 102 kDa and is encoded by a transcript of 3.8 kb at low, constitutive levels in many tissues. PRKCM phosphorylates protein kinase D (PKD). Inhibition of PKD activity prevents G protein β- and γ-mediated Golgi breakdown. PKD is recruited to the trans-Golgi network. PKD-mediated signaling regulates the formation of transport carriers from the trans-Golgi network in mammalian cells (Braon et al., Science, 295, 325-328 (2002)). PRKCM gene is believed to reside at chromosome 14q11.

PRKACA mediates many of the effects of cAMP in eukaryotic cells. PRKACA produces one of multiple subunits that form the cAMP-dependent protein kinase. The inactive cAMP-dependent protein kinase is a tetramer composed of 2 regulatory and 2 catalytic subunits. The cooperative binding of 4 molecules of cAMP dissociates the enzyme in a regulatory subunit dimer and 2 free active catalytic subunits. In humans 3 catalytic subunits are encoded by PRKACA, PRKACB, and PRKACG. The PRKACA gene is thought to reside at 19p13.1. Knocking out PRKACA in mice results in early postnatal death in the majority of the knockout mice, and knockout mice surviving exhibit stunted growth. In the surviving knockout mice, compensatory increases in PRKACB activity are observed.

ACVR1B is an activin A type 1B receptor precursor, serine-threonine protein kinase and belongs to the TGF-beta superfamily of structurally related signaling proteins. ACVR1B maps to chromosome 12q13 and has characteristics of a tumor suppressor gene.

CDK5R1 maps to chromosome 7q36. CDK5R1 is a 307 amino acid protein that is involved in cellular proliferation and neuronal pathway signaling. CDK5R1 knockout mice do not live long and have severe lesions in the neural system.

CDC42BPB is a 109-kD serine-threonine protein kinase that functions as a CDC42 effector in promoting cytoskeletal reorganization. CDC42BPB phosphorylates non-muscle myosin light chain that is required for actin-myosin contraction. This gene has been assigned to region 14q32.3.

MPP6 is a peripheral membrane-associated guanylate kinase. The 540-amino acid protein has a PDZ domain, a central SH3 domain, and a C-terminal GUK domain, which makes it similar to other members of the p55 MAGUK subfamily. MPP6 is believed to contain a protein 4.1 (EPB41)-binding domain with a characteristic tetra-lysine motif, a leucine zipper, and 2 phosphorylation sites. The protein is sometimes expressed from a 2.3-kb mRNA and/or a 4.2-kb transcript. Some studies have suggested that expression of MPP6 is highest in testis, and also expressed in ovary, prostate, thymus, small intestine, and several other tissues

### BRIEF SUMMARY OF THE INVENTION

The present invention provides a method of killing a hyperplastic, precancerous, and preferably cancer cells, by contacting the cancer cell with an inhibitor of a gene encoding one of the following:

(1) cyclin-dependent kinase 8 (CDK8),

(2) serine/threonine kinase 33 (STK33),

(3) protein kinase C-mu (PRKCM),

(4) cAMP-dependent protein kinase alpha (PRKACA),

(5) activin A receptor type 1B (ACVR1B),

(6) cyclin-dependent kinase 5 regulator 1 (CDK5R1); which is the 35 kDa regulator of CDK5,

(7) CDC42 binding protein kinase beta (DMPK-like) (CDC42BPB),

(8) palmitoylated 6 membrane protein (MAGUK p55 subfamily member 6) (MPP6), and

(9) CDC42 binding protein kinase alpha (DMPK-like) (CDC42BPA). The present invention also provides pharmaceutical compositions that include a therapeutically-effective quantity of an inhibitor of a gene expression of a gene selected from the group consisting of CDK8, STK33, PRKCM, PRKACA, ACVR1B, CDK5R1, CDC42BPB, MPP6, and CDC42BPA in a pharmaceutically acceptable carrier. Preferably, the composition is packaged in a unit-dose package, under sterile or aseptic conditions, and is packaged in light resistant packaging.

Also, provided is a method of identifying a cancer cell for any suitable use, including without limitation, detection of cancer, monitoring of therapeutic response, and monitoring relapse comprising detecting elevated expression of CDK8, STK33, PRKCM, PRKACA, ACVR1B, CDK5R1, CDC42BPB, MPP6, and CDC42BPA.

### DETAILED DESCRIPTION OF THE INVENTION

Expression of the genes CDK8, STK33, PRKCM, PRKACA, ACVR1B, CDK5R1, CDC42BPB, MPP6, and CDC42BPA have unexpectedly been found to be vital to the survival of cancer cells. Accordingly, it has now been found that cancer cells can be killed, and that cancer can be treated, by contacting a cancer cell with a cell-killing quantity, or a mammal with a therapeutically-effective quantity, of an inhibitor of the expression of a gene selected from the group consisting of CDK8, STK33, PRKCM, PRKACA, ACVR1B, CDK5R1, CDC42BPB, MPP6, and CDC42BPA.

Similarly, it has now been discovered that other methods of treating a mammal having cancer can be improved in a mammal in need thereof by including in the therapeutic regime the addition of an agent that impairs expression of a one or more genes selected from the group consisting of CDK8, STK33, PRKCM, PRKACA, ACVR1B, CDK5R1, CDC42BPB, MPP6, and CDC42BPA.

Additionally, it has now been discovered that the use of agents that impair the expression of a gene selected from the group CDK8, STK33, PRKCM, PRKACA, ACVR1B, CDK5R1, CDC42BPB, MPP6, and CDC42BPA can be administered to cancerous and non-cancerous cells in vitro so as to render them more sensitive to other cell killing compounds. This allows the rapid identification of agents that are reasonably expected to act synergistically with these gene inhibitors to impair growth or propagation of cancerous cells or kill cancerous cells. This also allows the identification of agents that can rescue cells that are dependent on the expression of these genes and that lack adequate expression of the product(s) of these genes.

Additionally, it has now been discovered that administering an inhibitor of gene expression of CDK8, STK33, PRKCM, PRKACA, ACVR1B, CDK5R1, CDC42BPB, MPP6, and CDC42BPA to a mixed population of cells comprising cancerous and non-cancerous cells can diminish the population of cancerous cells, and thereby enrich the population in non-cancerous cells. To enrich a population of cells in noncancerous cells when the population comprises both cancerous and non-cancerous cells, the mixture of cells is maintained under suitable conditions for cell survival for a suitable time (e.g., without limitation, for about 18 to about 120 hours, preferably 30 to 80 hours).

For example, the skilled artisan can selectively kill cancer cells in a population of human cells comprising human stem cells and cancer cells that are dependent for survival on gene expression of a gene selected from CDK8, STK33, PRKCM, PRKACA, ACVR1B, CDK5R1, CDC42BPB, MPP6, and CDC42BPA. By killing a portion of the cancer cells with an inhibitor of one or more of these genes, the remaining population, which optionally can be further purified by conventional methods, has a higher proportion of stem cells to cancer cells. In one particular embodiment, hematopoietic stem cells taken from a mammal are isolated from cancerous cells by the present inventive method and then administered to a mammal in need of hematopoietic stem cells (for example, because the mammal has previously undergone high dose radiation treatment to destroy its hematopoietic system).

Any suitable method of impairing or blocking the expression of these genes can be used. The cells can be from any mammal, such as horses, cats, mice, rats, rabbits, goats, sheep, cows, and humans. The mammal, however, is preferably a horse, dog, or cat, and more preferably is a human. Additionally, the hyperplastic, precancerous, and preferably cancerous cells can be treated in a mammal's body or first removed from a mammal's body and then killed.

Moreover, hyperplastic and cancerous cells can be removed from mixed cultures of cells, which mixtures contain undesirable hyperplastic cells that are dependent on the expression of a CDK8, a STK33, a PRKCM, a PRKACA, a ACVR1B, a CDK5R1, a CDC42BPB, a MPP6, or a CDC42BPA gene and desirable cells used either for biological research, or for the production of useful reagents [such as, without limitation, monoclonal antibodies, therapeutic growth factors (e.g., recombinant erythropoietin), and the like] can be enriched for desirable cells by administering to the mixture of cells a lethally-effective amount of an inhibitor of CDC42BPA (PK428) gene expression such that a portion of the CDC42BPA (PK428)-dependent cells are killed and the resulting mixture contains a higher proportion of desirable cells.

Hyperplasias generally refer to cells that exhibit abnormal and excessive growth in their normal location in a mammal's body, but do not generally exhibit microscopically evident morphological abnormalities that are thought to lead to cancer.

Precancerous cells can also be hyperplasias, but need not be hyperplasias. Precancerous cells have significant changes in cellular structure that can include (without limitation) chromosomal abnormalities (such as gene duplications, gene deletions, gene translocations, and microsatellite alterations), changes from the normal shape of the cell, changes in the ploidy of the cell, and abnormal expression of particular gene products. These changes tend to render precancerous cells particularly susceptible to additional changes that convert a precancerous cell into a cancerous cell.

The term “cancer” is understood in the art and is used broadly herein. Cancers are commonly divided into two groups that include carcinomas and sarcomas, but cells maintained in vitro that have the characteristics of cancer can also be referred to as cancerous cells. Cancerous cells are primarily defined by their ability to display abnormally invasive growth. Cancerous cells frequently also display one or more additional characteristics such as the ability to stimulate abnormal angiogenesis in normal cells, anchorage independent growth, chromosomal instability, and sometimes a capacity for invasive growth through organ barriers or into additional tissues.

The expression of the CDK8, STK33, PRKCM, PRKACA, ACVR1B, CDK5R1, CDC42BPB, MPP6, and CDC42BPA genes can be impaired or blocked by any suitable means. For example, (a) one or more of these genes can be modified in the genome of hyperplastic, precancerous, and cancerous cells of the mammal, (b) the processing or translation of the RNA product(s) of these genes can be impaired, blocked, or altered, (c) the function of the polypeptidyl product of these genes can be impaired or altered, and (d) the activity of these genes can be blocked by interfering with the gene function of CDK8, STK33, PRKCM, PRKACA, ACVR1B, CDK5R1, CDC42BPB, MPP6, or CDC42BPA gene. General methods for impairing the expression of specific genes by each of the foregoing methodologies are known to the skilled artisan.

In embodiments in which a gene is modified in the genome of the hyperplastic, precancerous, or cancerous cell, any suitable interruption of the gene function can be used. For example, the promoter may be silenced, e.g., via targeted methylation or other chemical derivation, DNA encoding the promoter or an RNA splice site can be removed or altered, mutations introducing missense, nonsense, or stop codons can be placed into the coding sequence or cause a frameshift deletion, and a portion of the genome can be exchanged with a sequence on an extrachromosomal vector.

In embodiments in which the processing or translation of the RNA product(s) of a gene can be impaired, blocked, or altered, any suitable method may be used. For example, the RNA product of a CDK8, STK33, PRKCM, PRKACA, ACVR1B, CDK5R1, CDC42BPB, MPP6, or CDC42BPA gene can be cleaved or rendered susceptible to rapid degradation, translation of the RNA can be blocked or reduced to lower the amount of the polypeptidyl product of a target gene product in the cell, preferably through the introduction of frameshift, or stop codons into the mRNA, the DNA encoding the RNA can be changed in order to introduce a heterologous polypeptide sequence of the polypeptidyl product of the target gene product thereby reducing the polypeptidyl product's activity, and specific inhibitors of translation can be contacted to the RNA.

In one embodiment of the present invention, the inhibitor is an antisense oligonucleotide. Antisense oligonucleotides are at least 12 nucleotides in length, preferably at least 20 nucleotides in length, and are optionally longer. As their name implies, antisense oligonucleotides are single-stranded reverse complements of target mRNAs and are designed to hybridize to the target mRNA. Antisense oligonucleotides can be composed of any suitable nucleic acid material. Typically, antisense oligonucleotides comprise a DNA polymer, however, peptidyl nucleic acids (PNAs), RNAs, and other nucleic acid moieties known in the art are usually suitable for use as antisense inhibitors of gene function. Antisense oligonucleotides can be carried in a pharmaceutically-acceptable carrier and administered in any suitable manner. Antisense oligonucleotides are preferably supplied as a sterile solution at a suitable dose. Administration of antisense oligonucleotides by a volumetric ambulatory fusion pump is among the preferred embodiments. Mani et al., Clin. Cancer Res., 8(4): 1042-1048 (2002) provides a useful example of the therapeutic use of antisense RNAs and some background information useful to the skilled artisan.

In another embodiment, the inhibitor is an siRNA. The design and use of siRNAs in general are known in the art. Commonly siRNAs comprise first RNA strand and second RNA strand, each of which is preferably of 21, 22, or 23 nucleotides in length. The strands are complementary to each other, such that when annealed in a dimeric form each strand has a 2-nucleotide 3′ overhang. The overhang residues need not be ribonucleotides; in fact deoxyribonucleotides and non-naturally occurring bases are among the chemical moieties that can be incorporated into the 3′-overhangs of the dimeric siRNA. The RNA is preferably selected such that the first RNA strand binds only to a CDK8, STK33, PRKCM, PRKACA, ACVR1B, CDK5R1, CDC42BPB, MPP6, or CDC42BPA gene, but this is not a necessary feature of the siRNA as long as the expression of at least one of these target genes is inhibited.

While not desiring to be bound by any particular theory, it is currently believed that the duplexed RNAs are incorporated into a protein complex called a RNA-induced silencing complex (RISC) which recognizes and cleaves the target mRNA. The siRNA can be delivered to the hyperplastic, precancerous, or cancerous cell by any suitable means. For example, the siRNA can be injected into the cell, placed on the cell in a suitable solvent (such as a carrier comprising dimethylsulfoxide or magic methyl). Similarly, cationic lipid systems, such as TransIT-TKO™ (Mirus, Madison, Wis.), GeneSilencer™ (GeneTherapySystems, San Diego, Calif.) or Lipofectamine (Invitrogen, Carlsbad, Calif.) can be used to facilitate the transfer of the siRNA into the hyperplastic, precancerous, or cancerous cell. Additionally, the siRNA can be delivered to cells in vivo. Multiple methods of delivering siRNA in vivo are known in the art. For example, Song et al. (Nat Med, published online (Feb. 10, 2003) doi:10.1038/nm828) and others (Caplen et al., Proc. Natl. Acad. Sci. (USA), 98, 9742-9747 (2001) and McCaffrey et al., Nature, 418, 38-39 (2002)) disclose that liver cells can be efficiently transfected by injection of the siRNA into a mammal's circulatory system. Viral vector-mediated siRNA delivery has been reported in xia et al., Nat. Biotechnol., 20, 1006-1010 (2002). Use of other nucleic acid delivery systems are also within the skill of the ordinarily skilled artisan.

Similarly, naked DNA or RNA molecules that are inhibitors of gene expression can be contacted to hyperplastic, precancerous, and preferably cancerous cells to kill these cells. When naked DNA or RNA is used it is preferably used in a form that is resistant to degradation such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to poly-lysine or transferrin. Nucleic acid can also be transported into cells by any of the various viral carriers, including but not limited to, retroviral vectors, vaccinia vectors, adeno-associated viral vectors (AAV), and adenoviral vectors.

In addition to killing hyperplastic, precancerous, or cancerous cells, the inhibitor of the target genes of the present invention can be admininistered to a mammal at risk of developing cancer. For example, the inhibitors can be administered to breast cancer patients who appear to have been successfully treated in order to prevent ocult tumor sites or micromatastases from growing into a clinical relapse.

Similarly, the inhibitors of the target genes can be administered to a mammal with cancer so as to treat a cancer, wherein the goal of such treatment is to slow progression of the cancer, or optionally, to prevent an increased load of tumor cells at a primary or peripheral tumor site.

A composition having the ability to inhibit the expression of a target gene can be assayed to determine its optimum therapeutic dosage alone or in combination with other inhibitors. Such assays are well known to those of skill in the art, and include without limitation tissue culture and animal models for various disorders that are treatable with such agents. For example, the Toxilight™ assay described in the Examples below can be usefully employed.

The skilled artisan will recognize that there are other assays and models for disease states available, including testing in humans. These assays can be used to measure the effectiveness of inhibitors of the target genes described above for a particular hyperplastic, precancerous, or preferably cancerous cell, and to determine the dosages for administration, with routine experimentation. Nonetheless, where the inhibitor is an siRNA any suitable amount of siRNA can be used. For example, from 5 pg to 100 μg of siRNA can be applied to a population of 10⁶ cells in vivo or in vitro.

Generally, similar or higher dosages will be applied when the inhibitor is applied systemically. Greater dosages will frequently be optimal when the cells to be killed are in a locus having high rates of fluid exchange or having conditions that accelerate deactivation or destruction of the inhibitor. Conversely, lower dosages can be applied when the inhibitor is applied with a targeting agent that directs or “targets” the inhibitor to the cell to be killed.

In accordance with the present invention, hyperplastic, precancerous, and preferably cancerous conditions in a mammal can be beneficially treated by impairing, or preferably blocking, the expression of a CDK8, STK33, PRKCM, PRKACA, ACVRIB, CDK5R1, CDC42BPB, NIPP6, or CDC42BPA gene by administering to the mammal a therapeutic quantity of a pharmaceutical composition that inhibits the activity of these genes.

The pharmaceutical composition includes a pharmaceutically-acceptable carrier and a therapeutically effective amount of an inhibitor of gene expression of a CDK8, STK33, PRKCM, PRKACA, ACVR1B, CDK5R1, CDC42BPB, MPP6, or CDC42BPA gene. The pharmaceutical composition preferably is packaged under aseptic or sterile conditions so as to obtain a sterile pharmaceutical composition. Additionally, the pharmaceutical composition is preferably packaged in unit dosages suitable for killing cancer cells and/or treating cancer. Moreover, the pharmaceutical composition is preferably packaged in light resistant packaging. The pharmaceutical composition can optionally also be packaged with instructions for administration to one or more mammals.

The inhibitor of the invention may also be used in combination with other therapeutic agents, for example (without limitation), chemotherapeutic compounds, antiemetics, and growth factors. When used with other chemotherapeutic agents, cancerous or precancerous cells are preferably more effectively killed. In the alternative, the optimal therapeutic dosage of the both the inhibitor of a target gene (as described above) and of the other chemotherapeutic agent are decreased to a level which results in the equivalent effectiveness of killing cancer cells as that with either agent applied alone at its optimum concentration. Although cancer cells are not more effectively killed, unwanted side effects (either in vivo or in vitro) are reduced. Especially when applied in vivo, the skilled artisan sometimes refers to this as increasing the therapeutic index.

The inhibitor can be contacted to a mammal or particular cells directly (i.e., alone) or preferably in a composition including a pharmaceutically acceptable carrier. Any suitable quantity of the inhibitor can be administered to the hyperplastic, precancerous, or cancerous cell, depending upon the location of the cell, the quantity of cells to be treated, whether the cell is growing in vitro or in vivo, whether the hyperplastic, precancerous, or cancerous cells are growing in an isolated location or intermixed with desirable cells. Additionally, when an inhibitor of the target gene (as described above) is administered to a mammal, the skilled artisan will consider the age, weight, gender, and general state of health of the mammal.

One of skill in the art will recognize that the toxicity for different inhibitors either alone, in combination with each other, or in combination with other pharmaceuticals can limit the maximum dose administered to a patient. Those of skill in the art may optimize dosage optimization for maximum benefits with minimal toxicity in a patient without undue experimentation using any suitable method. Additionally, the inhibitors of the present invention can be administered in vivo according to any of the methods described in exemplary texts, such as “Remington's Pharmaceutical Sciences” (8th and 15th Editions); the “Physicians' Desk Reference”, and the “Merck Index.”

The present invention also provides a pharmaceutical composition comprising a pharmaceutically acceptable carrier and a therapeutically effective amount at least one inhibitor of expression of at least one gene from the group consisting of CDK8, STK33, PRKCM, PRKACA, ACVR1B, CDK5R1, CDC42BPB, MPP6, and CDC42BPA. Any suitable carrier can be used in the pharmaceutical composition, which will depend in part on the particular means or route of administration, as well as other practical considerations. Such practical considerations include, but need not be limited to, providing a carrier suitable for the solubility of the inhibitor, and protection of the inhibitor from inactivation or degradation prior to delivery to target cells, tissues, and systems.

The pharmaceutically acceptable carriers described herein, for example, vehicles, excipients, adjuvants, or diluents, are well known to those who are skilled in the art and are readily available to the public. Accordingly, there are a wide variety of suitable formulations of the pharmaceutical composition of the present invention. The following formulations are exemplary and not necessarily meant to suggest the other formulations are not suitable.

Formulations that are injectable are among the preferred formulations. The requirements for effective pharmaceutical carriers for injectable compositions are well known to those of ordinary skill in the art (See Pharmaceutics and Pharmacy Practice, J. B. Lippincott Company, Philadelphia, Pa., Banker and Chalmers, eds., pages 238-250, (1982); ASHP Handbook on Injectable Drugs, Toissel, 4th ed., pages 622-630 (1986)). Such injectable compositions preferably can be administered intravenously or locally, i.e., at or near the site of a disease, or other condition in need of treatment.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and sterile suspensions that can include suspending agents, solubilizers, thickening agents, stabilizers, and preservatives. The target gene expression inhibitor can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, dimethylsulfoxide, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations, include petroleum, animal, vegetable, or synthetic oils. Specific examples of oils include peanut, soybean, sesame, cottonseed, corn, olive, petrolatum, and mineral.

Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-b-aminopropionates, and 2-alkyl-imidazoline quaternary ammonium salts, and (e) mixtures thereof.

The parenteral formulations will typically contain from about 0.0005% to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such formulations will typically range from about 5% by weight to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Topical formulations are well known to those of skill in the art and are suitable in the context of the present invention. Such formulations are typically applied to skin or other body surfaces.

Formulations suitable for oral administration can consist of (a) liquid solutions, such as an effective amount of the target gene expression inhibitor carried or suspended in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations can include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard-shelled or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

The target gene expression inhibitor useful in the context of the present invention, alone or in combination with other suitable components can be made into aerosol formulations to be administered via inhalation. These aerosol formulations can be placed into pressurized acceptable propellants, such as dichlorodifluoromethane, propane, nitrogen, and the like. They also may be formulated for non-pressured preparations, such as in a nebulizer or an atomizer. Such spray formulations are particularly suitable for spray application to mucosa.

Additionally, the target gene expression inhibitor can be made into suppositories by mixing with a variety of bases, such as emulsifying bases or water-soluble bases. Formulations suitable for vaginal and other administration can be presented as pessaries, tampons, creams, gels, pastes, foams, or spray formulas containing, in addition to the active ingredient, such carriers as are known in the art to be appropriate.

In addition to the above-described pharmaceutical compositions, the target gene expression inhibitor can be formulated as inclusion complexes, such as cyclodextrin inclusion complexes, or in liposomes (including modified liposomes such as pegylated and/or targeted liposomes.

The following example(s) further illustrate(s) the present invention but should not be construed as limiting its scope.

### EXAMPLES

### Example 1

The following example describes siRNAs, cell culture technique, and certain nucleic acid detection systems used in other examples presented herein.

The following examples use siRNAs designed in accordance with the rules suggested by Elbashir et al., Genes Dev, 15, 188-200 (2001). However, other methods of designing siRNAs are known and can be suitably used. In keeping with the Elbashir rules, the antisense strand of the siRNA is capable of hybridizing to the (N)₁₉ portion of a sequence of AA(N)₁₉, wherein each instance of N was independently selected from A, G, C, and T, and was the reverse complement of an mRNA sequence at least 100 nucleotides downstream of the translation start codon. The siRNAs also contain a 2 deoxynucleotide 3′ overhang (when the antisense strand of the siRNA is annealed to the sense strand of the siRNA duplex) which consists of dTdT. While any suitable GC content can be incorporated into the siRNA, the GC content of the siRNA duplexes used or referred to below was from 40% to 70%. Additionally, both strands of the siRNA were evaluated to ensure that the targeted sequence is not highly homologous to any non-targeted sequences known to exist in the genome of a treated cell. No sequences having 16 or more bases of complementarity were used in the following examples, although it should be appreciated that siRNAs with high homology to multiple sequences within a cell are also useful in the context of the present invention, even though non-specific siRNAs were not used in the following examples because of the potential to complicate evaluation of the data.

Human non-small cell lung carcinoma cells H1299 were cultured in RPMI-1640 medium obtained from Invitrogen, Inc. The RPMI medium was supplemented with 10% fetal bovine serum, and the H1299 cells were maintained at 37 degrees Celsius in air containing 5% carbon dioxide. 3 μl of a 20 μM solution of siRNA was mixed with 15 μl of TranIT-TKO™ reagent obtained from Mirus Corporation (Madison, Wis.) and incubated in RPMI for 20 minutes. This mixture was then transfected into the H1299 cells, which were in 2.5 ml of medium in 60-mm tissue culture dishes.

Total RNA was extracted from the transfected cells using Trizol™ (Invitrogen) and then purified on Qiagen™ RNeasy columns. TaqMan® Real Time QPCR was performed on an ABI Prism 7700™ obtained from Applied Biosystems. Reverse transcription and amplification employed 100 ng of total RNA.

### Example 2

This example shows that inhibition of the PK428 gene kills cancer cells.

H1299 cells were transfected with siRNAs that disrupt the expression of the PK428 gene. 0.375 μl of a 20 μM solution of siRNA was mixed with 0.7 μl of TranIT-TKO™ reagent obtained from Mirus Corporation (Madison, Wis.) and incubated in Opti-MEM (Invitrogen, Inc.) for 20 minutes. This mixture was then transfected into the H1299 cells, which were in 100 μl of RPMI medium in 96 well culture plates. Positive and negative control transfections were also performed. Cell death was assessed using the Toxilight™ BioAssay™. The Toxilight™ BioAssay was found to have a dynamic range well suited to the purposes of the following examples that employ it, and to provide suitably reproducible results.

The Toxilight™ BioAssay Kit is a bioluminescent, non-destructive assay designed to measure the release of adenylate kinase, which is released into the culture medium when cells die. The enzyme actively phosphorylates ADP to form ATP and the resultant ATP is then measured using firefly luciferase. As the level of cell rupture increases, the amount of light generated also increases.

The ability of siRNA to inhibit the expression of the CDC42BPA gene was confirmed by QPCR.

Cell killing was measured 72 hours after the siRNA was transfected into the H1299 cells. Data were obtained from samples in triplicate. As indicated by the Toxilight™ assay, siRNAs directed against CDC42BPA gene expression as well as the positive control reagent rapidly and effectively killed transfected H1299 cells, whereas the negative control reagent did not kill most of the transfected H1299 cells. Killing of H1299 cells achieved by inhibiting CDC42BPA expression was substantial and the signal generated by the assay was about one-half the signal (2.5-fold increase in light units) obtained with siRNA inhibitors of Eg5 (5-fold increase in light units) (a gene encoding a kinesin whose expression is known to be essential for viability of cancer cells) and “polo-like kinase 1” (“plk1”; 6 light units) (a gene encoding a cell cycle control kinase whose expression is known to be essential for viability of cancer cells).

When the siRNA targeted against CDC42BPA having the following structure was used:

| | |
|---|---|
| 5′ GGUGAUUGGUCGAGGAGCUdTdT 3′, | [SEQ ID NO: 1] |
| and | |
| 5′ AGCUCCUCGACCAAUCACCdTdT 3′, | [SEQ ID NO: 2] |

wherein A, U, G, and C are ribonucleotide bases, and dT is deoxythymidine then the majority of H1299 cells were killed within 72 hours.

The following siRNAs are also suitable inhibitors of CDC42BPA (or PK428) gene expression:

| | |
|---|---|
| 5′ AAUUCUGA AACGAUGCCC CdTdT 3′ | [SEQ ID NO: 3] |
| 5′ GGGGCAUC GUUUCAGAAU UdTdT 3′, | [SEQ ID NO: 4] |
| and | |
| 5′ CAUCGACU UGGUCAAAGU GdTdT 3′ | [SEQ ID NO: 5] |
| 5′ CACUUUGA CCAAGUCGAU GdTdT 3′ | [SEQ ID NO: 6] |
| 5′ AAGCUGACGAGUGAACUUGdTdT 3′ | [SEQ ID NO: 7] |
| 5′ CAAGUUCACUCGUCAGCUUdTdT 3′ | [SEQ ID NO: 8] |

however, the effectiveness of these latter siRNAs has not yet been measured.

Accordingly, this example shows that inhibition of CDC42BPA (or PK428) gene expression effectively kills cancer cells.

### Example 3

This example shows that inhibition of CDC42BPA (or PK428) kills multiple types of cancer cells.

786-O cells, which are derived from renal adenocarcinoma, were treated with the siRNA inhibitor of CDC42BPA used in Example 2. The cells were cultured similarly to the H1299 cells of Example 2 and assayed in a Toxilight™ assay. The data show that inhibition of CDC42BPA (PK428) expression killed cancer cells to an extent similar to that of H1299 cells. Specifically, a 1.5-fold increase in light units relative to negative control when the siRNA targeted against CDC42BPA was transfected into the 786-O cell line was observed.

### Example 4

This example shows that contacting cancerous cells with the siRNA inhibitor of CDC42BPA results in a decrease of CDC42BPA (or PK428) mRNA expression.

H1299 cells were treated as in Example 1. RNA was extracted from (1) cells transfected with the siRNA directed against CDC42BPA and from (2) cells not treated with the siRNA. The quantity of CDC42BPA RNA was measured in both cell samples. Expression of CDC42BPA RNA was 60% less in siRNA transfected H1299 cells than in non-transfected H1299 cells. Expression of CDC42BPA RNA also was 50% less in siRNA transfected 786-O cells than in non-transfected 786-O cells.

Thus, these data suggest that the cell death observed in cancer cells transfected with siRNA targeted against CDC42BPA results from inhibition of CDC42BPA (PK428) expression.

### Example 5

This example shows that CDC42BPA (PK428) is overexpressed in breast cancer tumors compared to non-cancerous breast tissue. This example also shows that CDC42BPA (PK428) expression is not significantly augmented in some other cancer tissues.

Comparative Quantitative PCR analysis of CDC42BPA mRNA expression in normal and tumor tissues was performed on normal and cancerous tissues taken from breast, lung, colon, and ovary. CDC42BPA expression at the mRNA level was elevated at least 2-5fold in 70% of the breast cancer tissues analyzed as compared to normal breast tissues. In contrast, CDC42BPA mRNA expression was not differentially expressed in lung, colon and ovary tumor tissues compared to the respective normal tissue.

Accordingly, breast cancer can be distinguished from non-cancerous breast cells and from non-breast cancers by determining if the degree of expression of CDC42BPA RNA in a test cell is elevated above the degree of expression expected in a normal cell of the type tested. Additionally, an inhibitor of CDC42BPA gene expression is particularly well suited to the treatment of breast cancer.

### Example 6

This example shows that inhibitors of CDC42BPA (PK428) expression cause perturbations in the S-phase of the cell cycle. Accordingly, this example also shows that co-administration of CDC42BPA expression inhibitors with agents or chemotherapeutics that have a principle effect on other cell cycle checkpoints or cell cycle phases between other checkpoints, can be administered with CDC42BPA inhibitors to create a synergistic therapeutic effect or to maintain therapeutic action while decreasing the amount of the other agent administered. That is, this example shows that co-administration of CDC42BPA expression inhibitors with agents or chemotherapeutics that have a principle effect on other cell cycle checkpoints can raise the “therapeutic index.”

H1299 cells were transfected with PK428 siRNA in accordance with previous methods. This resulted in approximately 7-10% increase in the number of cells in S-phase by 48 hours after transfection, and by 72 hours there was a marked decrease in DNA synthesis (i.e., growth arrest) compared to cells transfected with an siRNA that was designed not to interfere with any particular RNA (i.e., a “scrambled siRNA negative control”). Accordingly, this example shows that inhibition of CDC42BPA gene expression substantially interferes with progression through S-phase.

### Example 7

This example also shows that inhibition of the CDC42BPA gene expression kills cancer cells.

The conditions of Example 1 are used to grow H1299 cells, which are transfected with antisense RNAs directed against CDC42BPA gene expression. Cell viability is assessed using the Toxilight™ BioAssay™ as described in Example 2.

The ability of the antisense RNA to inhibit the expression of the CDC42BPA gene is preferably confirmed by QPCR.

Cell killing is measured 72 hours after the antisense RNAs are transfected into the target cells. The Toxilight™ assay indicates that antisense RNAs directed against CDC42BPA gene expression as well as the positive control reagents rapidly and effectively kill transfected H1299 cells and other cancerous cells, whereas the negative control reagent does not kill most of the transfected H1299 cells.

The antisense oligonucleotides can have any suitable sequence including without limitation:

| | |
|---|---|
| AGCTCCTCGA CCAATCACCT | [SEQ ID NO: 9] |
| GGGGCATCGT TTCAGAATTT | [SEQ ID NO: 10] |
| CACTTTGACCA AGTCGATGT | [SEQ ID NO: 11] |
| CAAGTTCACTC GTCAGCTTT | [SEQ ID NO: 12] |

Accordingly, this example also will show that inhibition of CDC42BPA gene expression effectively kills cancer cells.

### Example 8

This example shows how to generate antibody and antibody fragments useful in generating polypeptides of various classes useful in inhibiting the activity of the CDC42BPA gene (or any other target gene of the present invention). The antibodies can be contacted to CDC42BPA (or other target) gene products either intracellularly or under suitable conditions to the surface of a hyperplastic, precancerous, or preferably cancerous cell to inhibit CDC42BPA gene expression and treat a hyperplastic and preferably cancerous condition.

For the production of antibodies, various host animals may be immunized by injection with the CDC42BPA polypeptidyl gene product (or the polypeptidyl gene product of another target gene product), or a portion thereof including, but not limited to, portions of a the polypeptidyl gene product in a recombinant protein. Such host animals include but are not limited to rabbits, mice, rats, sheep, and other suitable animals. Similarly, immune responses can be raised in the mammal to be treated. Various adjuvants can be used to increase the immunological response, depending on the host species, including but not limited to Freund's (complete and/or incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and corynebacterium parvum.

Monoclonal antibodies can be prepared by using any suitable technique that provides for the production of antibody molecules by continuous cell lines in culture. These include but are not limited to the hybridoma technique originally described by Kohler and Milstein, Nature, 256, 495-497 (1975), the human B-cell hybridoma technique (Kosbor et al., Immunology Today, 4, 72 (1983), Cote et al., Proc. Natl. Acad. Sci., 80, 2026-2030 (1983)) and the EBV-hybridoma technique (Cole et al., 1985, MONOCLONAL ANTIBODIES AND CANCER THERAPY, Alan R. Liss, Inc., pp. 77-96). In addition, techniques developed for the production of “chimeric antibodies” (Morrison et al., Proc. Nat'l. Acad. Sci.(USA), 81, 6851-6855 (1984); Neuberger et al., Nature, 312:604-608 (1984); Takeda et al., Nature, 314, 452-454 (1985)) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be used to produce single chain antibodies specific to a target gene product.

Antibody fragments that recognize specific epitopes can be generated by known techniques. For example, such fragments include but are not limited to: the F(ab′)₂ fragments which can be produced by pepsin digestion of the antibody molecule and the FAb fragments which can be generated by reducing the disulfide bridges of the F(ab′)₂ fragments. Alternatively, FAb expression libraries may be constructed (Huse et al., Science, 246, 1275-1281(1989)) to allow rapid and easy identification of monoclonal FAb fragments with the desired specificity. Other methods of generating antibody-like fragments are also well understood in the art and can be used in the context of the present invention to create inhibitors of target gene expression that inhibit the expression of the target gene at the level of the polypeptidyl product.

The antibody or antibody fragment can be expressed within a target cell or contacted to the surface of the target cell under suitable conditions by conventional methods.

### Example 9

This example shows that small molecule inhibitors of CDC42BPA gene expression are effective in killing cancerous cells. This example also demonstrates that small molecule inhibitors of other target genes of the present invention are effective in killing cancerous cells.

Small molecules that interact with the polypeptidyl gene products of the target genes are among the preferred inhibitors of target gene expression. Chemical agents, referred to in the art as “small molecule” compounds are typically organic, non-polypeptidyl molecules having a molecular weight less than 10,000 Da, preferably less than 5,000 Da, more preferably less than 1,000 Da. This class of modulators includes chemically synthesized molecules, such as compounds from combinatorial chemical libraries. Synthetic compounds can be rationally designed or identified based on known or inferred properties of the protein product of the target genes or can be identified by screening compound libraries. Alternative appropriate modulators of this class are natural products, particularly secondary metabolites from organisms such as plants or fungi, which can also be identified by screening compound libraries for target gene expression inhibiting-activity. Methods for generating and obtaining compounds are well known in the art (See, e.g., Schreiber, Science, 151, 1964-1969 (2000); Radmann et al., Science, 151, 1947-1948 (2000)).

The cDNA of CDC42BPA, or optionally a portion of CDC42BPA such as the portion originally called PK428, is cloned into a yeast or bacterial expression vector. The expression vector is transfected into suitable cells under conditions selective for maintenance of the expression vector in the cells and conditional or unconditional expression of the protein in the cells. A library of small molecules is screened for the enhanced ability to bind to transfected cells as compared to non-transfected cells. Three compounds that preferentially bind to the transfected bacterial cells as compared to non-transfected bacterial cells are identified. These three compounds are applied to H1299 cells and kill H1299 cells more effectively than they kill non-cancerous lung small cells.

Thus, this example will show that small molecule inhibitors of CDC42BPA are effective at selectively killing cancerous cells, and in particular breast cancer cells.

### Example 10

This example will show another method by which CDC42BPA gene expression can be blocked so as to kill hyperplastic, precancerous, or preferably cancerous cells.

CDC42BPA gene expression is blocked by ribozyme molecules designed to cleave and destroy the mRNA in a target cell. The ribozyme molecules are optionally specific for the PK428 portion of the CDC42BPA gene and are designed according to principles generally well understood by those of skill in the art.

Ribozymes are RNA molecules that possess highly specific endoribonuclease activity. Hammerhead ribozymes comprise a hybridizing region that is complementary in nucleotide sequence to at least part of the target RNA, and a catalytic region that is adapted to cleave the target RNA. The hybridizing region contains nine (9) or more nucleotides. Therefore, the hammerhead ribozymes of the present invention have a hybridizing region that is complementary to the mRNA sequence of the PK428 gene and is at least nine nucleotides in length. The construction and production of such ribozymes is well known in the art and is described more fully in Haseloff and Gerlach, Nature, 334:585-591 (1988).

The ribozymes of the present invention also include RNA endoribonucleases (sometimes called “Cech-type ribozymes”) such as the one which occurs naturally in Tetrahymena Thermophila (known as the IVS, or L-19 IVS RNA) and which has been extensively described by Cech et al.: (Zaug, et al., Science, 224, 574-578 (1984); Zaug et al., Science, 231, 470-475 (1986); Zaug, et al., Nature, 324, 429-433 (1986); International patent application No. WO 88/04300 (University Patents); Been et al., Cell, 47, 207-216 (1986)). The “Cech-type endoribonucleases” have an eight base pair active site that hybridizes to a target RNA sequence and cleave the target RNA. The invention encompasses those Cech-type ribozymes that target eight base-pair active site sequences that are present in any of the target genes described above in the present invention.

In another alternative, oligonucleotides designed to hybridize to the 5′ region of the CDC42BPA gene (including the region upstream of the coding sequence) and form triple helix structures through Hoogstein (non-Watson & Crick) base pairing are used to impair transcription of the CDC42BPA gene.

Accordingly, this example will show yet another way of selectively killing hyperplastic, precancerous, or and preferably cancerous cells, including without limitation the treatment of cancer in a mammal in need thereof.

### Example 11

This example shows that inhibition of gene expression of CDK8, STK33, PRKCM, PRKACA, ACVR1B, CDK5R1, CDC42BPB, and MPP6 kills hyperplastic, precancerous, and in particular cancerous cells. siRNAs directed against each of these genes were transfected into H1299 cells, and 786-O cells as described above. The following table shows the degree of cancerous cell killing achieved by inhibiting each of these cells.

TABLE 1

siRNAs used in this example. (all strands have a dTdT at their 3′ end
indicated as dT below)

| | | | | |
|---|---|---|---|---|
| Gene Name or | | | | |
| Locus (GenBank | siRNA sequence | SEQ ID | siRNA sequence | SEQ ID |
| Accession No.) | (sense strand of duplex) | NO: | (anti-sense strand of duplex) | NO: |

| | | | | |
|---|---|---|---|---|
| CDK8 (NM_001260) | AGCCAAGAGG AAAGAUGGGdT dT | 13 | CCCAUCUUUC CUCUUGGCUdT dT | 14 |
| | GCGAAUUACC UCAGAACAGdT dT | 15 | CUGUUCUGAG GUAAUUCGCdT dT | 16 |
| | AGGUGUUUCU GUCUCAUGCdT dT | 17 | GCAUGAGACA GAAACACCUdT dT | 18 |
| | UAGAAGGAAC UGGGAUCUCdT dT | 19 | GAGAUCCCAG UUCCUUCUAdT dT | 20 |
| STK33 (NM_030906) | AACAAGGGUU CCUCCAGUUdT dT | 21 | AACUGGAGGA ACCCUUGUUdT dT | 22 |
| | AGUCUCGCAU CAGCUAUAGdT dT | 23 | CUAUAGCUGA UGCGAGACUdT dT | 24 |
| | GUUACUUGAA CGAGAGGUGdT dT | 25 | CACCUCUCGU UCAAGUAACdT dT | 26 |
| | CGAGAGGUGA ACAUUCUGAdT dT | 27 | UCAGAAUGUU CACCUCUCGdT dT | 28 |
| PRKCM (NM_002742) | AACAUCCUUC AGCUGGUGAdT dT | 29 | UCACCAGCUG AAGGAUGUUdT dT | 30 |
| | GGCGAUCUUA UUGAAGUGGdT dT | 31 | CCACUUCAAU AAGAUCGCCdT dT | 32 |
| | GAAGCAAUGG UCCAAGAUGdT dT | 33 | CAUCUUGGAC CAUUGCUUCdT dT | 34 |
| | AUACCCAACA AUUGCAGCGdT dT | 35 | CGCUGCAAUU GUUGGGUAUdT dT | 36 |
| PRKACA (NM_002730) | CAGAUCGAAC ACACCCUGAdT dT | 37 | UCAGGGUGUG UUCGAUCUGdT dT | 38 |
| | GAAGGGCAGC GAGCAGGAGdT dT | 39 | CUCCUGCUCG CUGCCCUUCdT dT | 40 |
| | GGGCAGCGAG CAGGAGAGCdT dT | 41 | GCUCUCCUGC UCGCUGCCCdT dT | 42 |
| | CCUUCCUUUC GGAGUAAUCdT dT | 43 | GAUUACUCCG AAAGGAAGGdT dT | 44 |
| ACVR1B (NM_004302) | CGAUACAUGG CCCCUGAAGdT dT | 45 | CUUCAGGGGC CAUGUAUCGdT dT | 46 |
| | GACGUGAAGA UCUAACUGCdT dT | 47 | GCAGUUAGAU CUUCACGUCdT dT | 48 |
| | GAUGAUGCGA GAGUGUUGGdT dT | 49 | CCAACACUCU CGCAUCAUCdT dT | 50 |
| | CUGCUCCCUC UCUCCACACdT dT | 51 | GUGUGGAGAGA GGGAGCAGdT dT | 52 |
| CDK5R1 (NM_003885) | CGCCAAGGAC AAGAACCUGdT dT | 53 | CAGGUUCUUG UCCUUGGCGdT dT | 54 |
| | UGAGAACCUG AAGAAGUCGdT dT | 55 | CGACUUCUUC AGGUUCUCAdT dT | 56 |
| | GAAGAACUCC AAGAAGGUGdT dT | 57 | CACCUUCUUG GAGUUCUUCdT dT | 58 |
| | CAGCAGCUAC CAGAACAACdT dT | 59 | GUUGUUCUGG UAGCUGCUGdT dT | 60 |
| CDC42BPB (NM_006035) | GCGAAGGACC UCAUCCAGAdT dT | 61 | UCUGGAUGAG GUCCUUCGCdT dT | 62 |
| | GCUUACGAGA GGAGGAUUCdT dT | 63 | GAAUCCUCCU CUCGUAAGCdT dT | 64 |
| | CUCAAAGAUG CCCAUCAGCdT dT | 65 | GCUGAUGGGC AUCUUUGAGdT dT | 66 |
| | CUUCGACGUG GAUGACGACdT dT | 67 | GUCGUCAUCC ACGUCGAAGdT dT | 68 |
| MPP6 (NM_016447) | GGCUCAUGAG ACGCUAGAAdT dT | 69 | UUCUAGCCUC UCAUGAGCCdT dT | 70 |
| | GUUUGUGUCA CGAUCUGAGdT dT | 71 | CUCAGAUCGU GACACAAACdT dT | 72 |
| | GAUGAAAAAG AUGGCCAGGdT dT | 73 | CCUGGCCAUC UUUUUCAUCdT dT | 74 |
| | AUGUGGCAGA AUUGGUUGGdT dT | 75 | CCAACCAAUU CUGCCACAUdT dT | 76 |

TABLE 2
Inhibition of target genes results in death of cancer cells.

| | Increase in ToxiLight rel. | Increase in ToxiLight rel. |
|---|---|---|
| | light units in H1299 cells | light units in 786-O cells |
| negative control | no increase | no increase (i.e., baseline) |
| | (i.e., baseline) | |
| positive controls | 5-6 fold | 2-fold |
| CDK8 | 1.7-fold | 1.3-fold |
| STK33 | 4.8-fold | 1.4-fold |
| PRKCM | 2.7-fold | 2.7-fold |
| PRKACA | 2.7-fold | 1.5-fold |
| ACVR1B | 1.7-fold | 1.6-fold |
| CDK5R1 | 4.4-fold | 1.3-fold |
| CDC42BPB | 9.3-fold | 1.6-fold |
| MPP6 | 6.3-fold | 1.3-fold |

TABLE 3
Inhibition of mRNA expression relative to controls
achieved by transfecting the siRNAs listed in Table 1.

| | |
|---|---|
| Relative mRNA | Suppression of |
| levels in | mRNA levels (from |
| untransfected | controls) by siRNA in: |

| | H1299 cells | H1299 cells | 786-O cells |
|---|---|---|---|
| CDK8 | 100 | 65% | 5% |
| STK33 | 100 | 60% | 40% |
| PRKCM | 100 | 40% | 90% |
| PRKACA | 100 | 60% | ND |
| ACVR1B | 100 | 25% | 10% |
| CDK5R1 | 100 | ND | 90% |
| CDC42BPB | 100 | 60% | 70% |
| MPP6 | 100 | 70% | 50% |

### Example 12

This example provides antisense oligonucleotides that will be useful in the inhibition of the target genes of the present invention.

TABLE 4
Antisense oligonucleotide inhibitors.

| | | |
|---|---|---|
| Gene Name (Locus) | Antisense Sequence | SEQ ID NO: |

| | | |
|---|---|---|
| CDK8 | CCCATCTTTCCTCTTGGCTT | 77 |
| | CTGTTCTGAGGTAATTCGCT | 78 |
| | GCATGAGACAGAAACACCTT | 79 |
| | GAGATCCCAGTTCCTTCTAT | 80 |
| STK33 | AACTGGAGGAACCCTTGTTT | 81 |
| | CTATAGCTGATGCGAGACTT | 82 |
| | CACCTCTCGTTCAAGTAACT | 83 |
| | TCAGAATGTTCACCTCTCGT | 84 |
| PRKCM | TCACCAGCTGAAGGATGTTT | 85 |
| | CCACTTCAATAAGATCGCCT | 86 |
| | CATCTTGGACCATTGCTTCT | 87 |
| | CGCTGCAATTGTTGGGTATT | 88 |
| PRKACA | TCAGGGTGTGTTCGATCTG | 89 |
| | CTCCTGCTCGCTGCCCTTC | 90 |
| | GCTCTCCTGCTCGCTGCCC | 91 |
| | GATTACTCCGAAAGGAAGG | 92 |
| ACVR1B | CTTCAGGGGCCATGTATCG | 93 |
| | GCAGTTAGATCTTCACGTC | 94 |
| | CCAACACTCTCGCATCATC | 95 |
| | GTGTGGAGAGAGGGAGCAG | 96 |
| CDK5R1 | CAGGTTCTTGTCCTTGGCG | 97 |
| | CGACTTCTTCAGGTTCTCA | 98 |
| | CACCTTCTTGGAGTTCTTC | 99 |
| | GTTGTTCTGGTAGCTGCTG | 100 |
| CDC42BPB | TCTGGATGAGGTCCTTCGCT | 101 |
| | GAATCCTCCTCTCGTAAGCT | 102 |
| | GCTGATGGGCATCTTTGAGT | 103 |
| | GTCGTCATCCACGTCGAAGT | 104 |
| MPP6 | TTCTAGCCTCTCATGAGCCT | 105 |
| | CTCAGATCGTGACACAAACT | 106 |
| | CCTGGCCATCTTTTTCATCT | 107 |
| | CCAACCAATTCTGCCACATT | 108 |
| CDC42BPA (PK428) | AGCTCCTCGACCAATCACCT | 109 |
| | GGGGCATCGTTTCAGAATTT | 110 |
| | CACTTTGACCAAGTCGATGT | 111 |
| | CAAGTTCACTCGTCAGCTTT | 112 |

### Example 13

This example gives the sequences of the mRNAs (SEQ ID NOS: 113-121) encoded by the target genes of the present invention. The skilled artisan will appreciate that minor sequence variations may occur between organisms and individuals in these genes and that occasional errors can be present. Nonetheless, the skilled artisan readily will be able to generate inhibitors of the target genes and also of the mRNAs of the target genes irrespective of whether some errors are present in the following sequences.

CDK8 mRNA accession no. NM_{—}001260 (gi:4502744)

| | |
|---|---|
| GGGCTCCGGCCTCAGAGGCTGTGACAATGGACTATGACTTTAAAGTGAAGCTGAGCAGCGAGCGGGAGCG | [SEQ ID NO: 113] |
| GGTCGAGGACCTGTTTGAATACGAGGGCTGCAAAGTTGGCCGAGGCACTTATGGTCACGTCTACAAAGCC | |
| AAGAGGAAAGATGGGAAGGATGATATAGACTATGCTTTAAAACAAATAGAAGGAACTGGGATCTCTATGT | |
| CGGCATGTAGAGAAATAGCATTACTTCGAGAGCTTAAGCATCCAAACGTCATTTCTCTTCAAAAGGTGTT | |
| TCTGTCTCATGCTGATAGGAAGGTGTGGCTTCTGTTTGACTATGCTGAACATGACCTCTGGCATATAATC | |
| AAGTTTCACAGAGCTTCTAAAGCAAACAAGAAGCCAGTTCAGTTACCTCGGGGAATGGTGAAGTCACTAT | |
| TATATCAGATCCTAGATGGTATTCACTACCTGCATGCTAACTGGGTGTTGCACAGAGATTTGAAACCTGC | |
| TAATATTTTAGTTATGGGTGAAGGTCCTGAGCGAGGAAGAGTAAAAATTGCTGACATGGGCTTTGCCCGA | |
| TTATTTAATTCACCTTTGAAGCCTTTAGCAGATTTGGATCCAGTGGTTGTTACATTCTGGTACCGAGCCC | |
| CTGAACTACTTCTTGGAGCAAGGCATTATACCAAAGCTATTGATATTTGGGCTATAGGGTGTATATTTGC | |
| AGAACTACTAACGTCAGAACCAATATTTCACTGTCGACAAGAGGACATCAAAACTAGTAATCCTTATCAC | |
| CATGACCAGCTGGACAGAATATTCAATGTAATGGGATTTCCTGCAGATAAAGATTGGGAAGATATAAAAA | |
| AGATGCCTGAACATTCAACATTAATGAAAGATTTCAGAAGAAATACGTATACCAACTGCAGCCTTATCAA | |
| GTATATGGAAAAACATAAAGTTAAACCAGATAGTAAAGCATTCCACTTGCTTCAGAAGCTGCTTACCATC | |
| GACCCAATAAAGCGAATTACCTCAGAACAGGCTATGCAGGACCCCTATTTCTTAGAAGACCCACTTCCTA | |
| CATCAGACGTTTTTGCCGGTTGTCAAATCCCTTACCCAAAACGAGAATTTTTAACGGAAGAAGAACCTGA | |
| TGACAAAGGAGACAAAAAGAACCAGCAGCAGCAGCAGGGCAATAACCACACTAATGGAACTGGCCACCCA | |
| GGGAATCAAGACAGCAGTCACACACAGGGACCCCCGTTGAAGAAAGTGAGAGTTGTTCCTCCTACCACTA | |
| CCTCAGGTGGACTTATCATGACCTCAGACTATCAGCGTTCCAATCCACATGCTGCCTATCCCAACCCTGG | |
| ACCAAGCACATCACAGCCGCAGAGCAGCATGGGATACTCAGCTACCTCCCAGCAGCCTCCACAGTACTCA | |
| CATCAGACACATCGGTACTGAGCTGCATCGGAATCTTGTCCATGCACTGTTGCGAATGCTGCAGGGCTGA | |
| CTGTGCAGCTCTCTGCGGGAACCTGGTATGGGCCATGAGAATGTACTGTACAACCACATCTTCAAAATGT | |
| CCAGTAGCCAAGTTCCACCACTTTTCACAGATTGGGGTAGTGGCTTCCAAGTTGTACCTATTTTGGAGTT | |
| AGACTTGAAAAGAAAGTGCTAGCACAGTTTGTGTTGTGGATTTGCTACTTCCATAGTTTACTTGACATGG | |
| TTCAGACTGACCAATGCATTTTTTTCAGTGACAGTCTGTAGCAGTTGAAGCTGTGAATGTGCTAGGGGCA | |
| AGCATTTGTCTTTGTATGTGGT | |

STK33 mRNA GenBank Accession No. NM_{—}030906 (gi:23943881)

| | |
|---|---|
| ATGTACTCCCAATTACTTCTGGAAGTTTCTCAAAGTACTCCTTTATATATACTGCAGAGTGTATTTTTCT | [SEQ ID NO: 114] |
| TCCTCCTCAACTGAGATCTTTCCAACTTGCCACCATGCAGCTGCCAATGGTCCTAGTTAAGTAAAATGCT | |
| GCCATACCTATTTTAGACTCAGGGAAAAATAGCACCCACTCATTTTTATTTTTGCTCAATATAAAAATGA | |
| GGATACTTATGAGGATACTTAAACTTTTAGGATTAGCTAGTTTTCTAAAAATCGAATTATTCACTCCTTT | |
| GTAAAGTATGTAATAGGAATTTGCTCTAATAATCAATAGATTAAGGTTTAAAATTTGAAACCATAGTAAT | |
| GTATGTTTAACACCAATATTTTAAGCCTTTTTAAAAACCACAACCCACATTAAGAAATACATTTCATACT | |
| TTCCAAGGAGGTATGCTAAATATTATCTCTTTGATTCTACTTTATTTTTAAAAAGTGGTATCAACCCACA | |
| AAATGGATTTCATAACCCACTACGCAGTTTGATAAGATGCTGTTTTAGACCATGCTTTTCACCAGTTTTG | |
| TGGTCCTATTTTGTCCTTTTCATGTCTATACAGGATGCTTCTAGTGCTAGTTGCTAGCTTTTCTCTGATT | |
| TCCAGGATGGTAATAGGTTAAGAATTTCTCTAAATGGTTATTTCTTTTCTTTCTGCAGCTCTCACGTGTG | |
| AATATGTGTCTAGTGCATCCTTAACCTGAGGACTTCACCAGTTCGAAATTACAGTTTTCACCATCAACTA | |
| CCTTATCCTTTTTGGCCTGGTTTTCTTCCTCAAACAGTGGAAACATTTTTAAAGTTGCTTTTGTTGCAGA | |
| GTTAAACAAATGGCTGATAGTGGCTTAGATAAAAAATCCACAAAATGCCCCGACTGTTCATCTGCTTCTC | |
| AGAAAGATGTACTTTGTGTATGTTCCAGCAAAACAAGGGTTCCTCCAGTTTTGGTGGTGGAAATGTCACA | |
| GATATAACCTCCAGGAAAGATTTGCCCTCAAGAACCTCAAATGTAGAGAGAAAAGCATCTCAGCAACAAT | |
| GGGGTCGGGGCAACTTTACAGAAGGAAAAGTTCCTCACATAAGGATTGAGAATGGAGCTGCTATTGAGGA | |
| AATCTATACCTTTGGAAGAATATTGGGAAAAGGGAGCTTTGGAATAGTCATTGAAGCTACAGACAAGGAA | |
| ACAGAAACGAAGTGGGCAATTAAAAAAGTGAACAAAGAAAAGGCTGGAAGCTCTGCTGTGAAGTTACTTG | |
| AACGAGAGGTGAACATTCTGAAAAGTGTAAAACATGAACACATCATACATCTGGAACAAGTATTTGAAAC | |
| GCCAAAGAAAATGTACCTTGTGATGGAGCTTTGTGAGGATGGAGAACTCAAAGAAATTCTGGATAGGAAA | |
| GGGCATTTCTCAGAGAATGAGACAAGGTGGATCATTCAAAGTCTCGCATCAGCTATAGCATATCTTCACA | |
| ATAATGATATTGTACATAGAGATCTGAAACTGGAAAATATAATGGTTAAAAGCAGTCTTATTGATGATAA | |
| CAATGAAATAAACTTAAACATAAAGGTGACTGATTTTGGCTTAGCGGTGAAGAAGCAAAGTAGGAGTGAA | |
| GCCATGCTGCAGGCCACATGTGGGACTCCTATCTATATGGCCCCTGAAGTTATCAGTGCCCACGACTATA | |
| GCCAGCAGTGTGACATTTGGAGCATAGGCGTCGTAATGTACATGTTATTACGTGGAGAACCACCCTTTTT | |
| GGCAAGCTCAGAAGAGAAGCTTTTTGAGTTAATAAGAAAAGGAGAACTACATTTTGAAAATGCAGTCTGG | |
| AATTCCATAAGTGACTGTGCTAAAAGTGTTTTGAAACAACTTATGAAAGTAGATCCTGCTCACAGAATCA | |
| CAGCTAAGGAACTACTAGATAACCAGTGGTTAACAGGCAATAAACTTTCTTCGGTGAGACCAACCAATGT | |
| ATTAGAGATGATGAAGGAATGGAAAAATAACCCAGAAAGTGTTGAGGAAAACACAACAGAAGAGAAGAAT | |
| AAGCCGTCCACTGAAGAAAAGTTGAAAAGTTACCAACCCTGGGGAAATGTCCCTGATGCCAATTACACTT | |
| CAGATGAAGAGGAGGAAAAACAGTCTACTGCTTATGAAAAGCAATTTCCTGCAACCAGTAAGGACAACTT | |
| TGATATGTGCAGTTCAAGTTTCACATCTAGCAAACTCCTTCCAGCTGAAATCAAGGGAGAAATGGAGAAA | |
| ACCCCTGTGACTCCAAGCCAAGGAACAGCAACCAAGTACCCTGCTAAATCCGGCGCCCTGTCCAGAACCA | |
| AAAAGAAACTCTAAGGTTCCCTCCAGTGTTGGACAGTACAAAAACAAAGCTGCTCTTGTTAGCACTTTGA | |
| TGAGGGGGTAGGAGGGGAAGAAGACAGCCCTATGCTGAGCTTGTAGCCTTTTAGCTCCACAGAGCCCCGC | |
| CATGTGTTTGCACCAGCTTAAAATTGAAGCTGCTTATCTCCAAAGCAGCATAAGCTGCACATGGCATTAA | |
| AGGACAGCCACCAGTAGGCTTGGCAGTGGGCTGCAGTGGAAATCAACTCAAGATGTACACGAAGGTTTTT | |
| TAGGGGGGCAGATACCTTCAATTTAAGGCTGTGGGCACACTTGCTCATTTTTACTTCAAATTCTTATGTT | |
| TAGGCACAGCTATTTATAGGGGAAAACAAGAGGCCAAATATAGTAATGGAGGTGCCAAATAATTATGTGC | |
| ACTTTGCACTAGAAGACTTTGTTAGAAAATTACTAATAAACTTGCCATACGTATTACAGCAGAAGTGCTT | |
| CAGTCATTCACATGTGTTCGTGAGATTTTAGGTTGCTATAGATTGTTTAAGACAGCTTATTTTAAATGTA | |
| GAAAAATAGGAGATTTTGTAACTGCTTGCCATTAACTTGCTGCTAAATTCCCAA | |

PRKCM mRNA. GenBank Accession No. NM_{—}002742 (gi:4506074)

| | |
|---|---|
| GAATTCCTTCTCTCCTCCTCCTCGCCCTTCTCCTCGCCCTCCTCCTCCTCCTCGCCCTCCCCTCCCGATC | [SEQ ID NO: 115] |
| CTCATCCCCTTGCCCTCCCCAGCCCAGGGACTTTTCCGGAAAGTTTTTTATTTTCCGTCTGGGCTCTCGG | |
| AGAAAGAAGCTCCTGGCTCAGCGGCTGCAAAACTTTCCTGCTGCCGCGCCGCCAGCCCCCGCCCTCCGCT | |
| GCCCGGCCCTGCGCCCCGCCGAGCGATGAGCGCCCCTCCGGTCCTGCGGCCGCCCAGTCCGCTGCTGCCC | |
| GTGGCGGCGGCAGCTGCCGCAGCGGCCGCCGCACTGGTCCCAGGGTCCGGGCCCGGGCCCGCGCCGTTCT | |
| TGGCTCCTGTCGCGGCCCCGGTCGGGGGCATCTCGTTCCATCTGCAGATCGGCCTGAGCCGTGAGCCGGT | |
| GCTGCTGCTGCAGGACTCGTCCGGGGACTACAGCCTGGCGCACGTCCGCGAGATGGCTTGCTCCATTGTC | |
| GACCAGAAGTTCCCTGAATGTGGTTTCTACGGAATGTATGATAAGATCCTGCTTTTTCGCCATGACCCTA | |
| CCTCTGAAAACATCCTTCAGCTGGTGAAAGCGGCCAGTGATATCCAGGAAGGCGATCTTATTGAAGTGGT | |
| CTTGTCACGTTCCGCCACCTTTGAAGACTTTCAGATTCGTCCCCACGCTCTCTTTGTTCATTCATACAGA | |
| GCTCCAGCTTTCTGTGATCACTGTGGAGAAATGCTGTGGGGGCTGGTACGTCAAGGTCTTAAATGTGAAG | |
| GGTGTGGTCTGAATTACCATAAGAGATGTGCATTTAAAATACCCAACAATTGCAGCGGTGTGAGGCGGAG | |
| AAGGCTCTCAAACGTTTCCCTCACTGGGGTCAGCACCATCCGCACATCATCTGCTGAACTCTCTACAAGT | |
| GCCCCTGATGAGCCCCTTCTGCAAAAATCACCATCAGAGTCGTTTATTGGTCGAGAGAAGAGGTCAAATT | |
| CTCAATCATACATTGGACGACCAATTCACCTTGACAAGATTTTGATGTCTAAAGTTAAAGTGCCGCACAC | |
| ATTTGTCATCCACTCCTACACCCGGCCCACAGTGTGCCAGTACTGCAAGAAGCTTCTGAAGGGGCTTTTC | |
| AGGCAGGGCTTGCAGTGCAAAGATTGCAGATTCAACTGCCATAAACGTTGTGCACCGAAAGTACCAAACA | |
| ACTGCCTTGGCGAAGTGACCATTAATGGAGATTTGCTTAGCCCTGGGGCAGAGTCTGATGTGGTCATGGA | |
| AGAAGGGAGTGATGACAATGATAGTGAAAGGAACAGTGGGCTCATGGATGATATGGAAGAAGCAATGGTC | |
| CAAGATGCAGAGATGGCAATGGCAGAGTGCCAGAACGACAGTGGCGAGATGCAAGATCCAGACCCAGACC | |
| ACGAGGACGCCAACAGAACCATCAGTCCATCAACAAGCAACAATATCCCACTCATGAGGGTAGTGCAGTC | |
| TGTCAAACACACGAAGAGGAAAAGCAGCACAGTCATGAAAGAAGGATGGATGGTCCACTACACCAGCAAG | |
| GACACGCTGCGGAAACGGCACTATTGGAGATTGGATAGCAAATGTATTACCCTCTTTCAGAATGACACAG | |
| GAAGCAGGTACTACAAGGAAATTCCTTTATCTGAAATTTTGTCTCTGGAACCAGTAAAAACTTCAGCTTT | |
| AATTCCTAATGGGGCCAATCCTCATTGTTTCGAAATCACTACGGCAAATGTAGTGTATTATGTGGGAGAA | |
| AATGTGGTCAATCCTTCCAGCCCATCACCAAATAACAGTGTTCTCACCAGTGGCGTTGGTGCAGATGTGG | |
| CCAGGATGTGGGAGATAGCCATCCAGCATGCCCTTATGCCCGTCATTCCCAAGGGCTCCTCCGTGGGTAC | |
| AGGAACCAACTTGCACAGAGATATCTCTGTGAGTATTTCAGTATCAAATTGCCAGATTCAAGAAAATGTG | |
| GATATCAGCACAGTATATCAGATTTTTCCTGATGAAGTACTGGGTTCTGGACAGTTTGGAATTGTTTATG | |
| GAGGAAAACATCGTAAAACAGGAAGAGATGTAGCTATTAAAATCATTGACAAATTACGATTTCCAACAAA | |
| ACAAGAAAGCCAGCTTCGTAATGAGGTTGCAATTCTACAGAACCTTCATCACCCTGGTGTTGTAAATTTG | |
| GAGTGTATGTTTGAGACGCCTGAAAGAGTGTTTGTTGTTATGGAAAAACTCCATGGAGACATGCTGGAAA | |
| TGATCTTGTCAAGTGAAAAGGGCAGGTTGCCAGAGCACATAACGAAGTTTTTAATTACTCAGATACTCGT | |
| GGCTTTGCGGCACCTTCATTTTAAAAATATCGTTCACTGTGACCTCAAACCAGAAAATGTGTTGCTAGCC | |
| TCAGCTGATCCTTTTCCTCAGGTGAAACTTTGTGATTTTGGTTTTGCCCGGATCATTGGAGAGAAGTCTT | |
| TCCGGAGGTCAGTGGTGGGTACCCCCGCTTACCTGGCTCCTGAGGTCCTAAGGAACAAGGGCTACAATCG | |
| CTCTCTAGACATGTGGTCTGTTGGGGTCATCATCTATGTAAGCCTAAGCGGCACATTCCCATTTAATGAA | |
| GATGAAGACATACACGACCAAATTCAGAATGCAGCTTTCATGTATCCACCAAATCCCTGGAAGGAAATAT | |
| CTCATGAAGCCATTGATCTTATCAACAATTTGCTGCAAGTAAAAATGAGAAAGCGCTACAGTGTGGATAA | |
| GACCTTGAGCCACCCTTGGCTACAGGACTATCAGACCTGGTTAGATTTGCGAGAGCTGGAATGCAAAATC | |
| GGGGAGCGCTACATCACCCATGAAAGTGATGACCTGAGGTGGGAGAAGTATGCAGGCGAGCAGCGGCTGC | |
| AGTACCCCACACACCTGATCAATCCAAGTGCTAGCCACAGTGACACTCCTGAGACTGAAGAAACAGAAAT | |
| GAAAGCCCTCGGTGAGCGTGTCAGCATCCTCTGAGTTCCATCTCCTATAATCTGTCAAAACACTGTGGAA | |
| CTAATAAATACATACGGTCAGGTTTAACATTTGCCTTGCAGAACTGCCATTATTTTCTGTCAGATGAGAA | |
| CAAAGCTGTTAAACTGTTAGCACTGTTGATGTATCTGAGTTGCCAAGACAAATCAACAGAAGCATTTGTA | |
| TTTTGTGTGACCAACTGTGTTGTATTAACAAAAGTTCCCTGAAACACGAAACTTGTTATTGTGAATGATT | |
| CATGTTATATTTAATGCATTAAACCTGTCTCCACTGTGCCTTTGCAAATCAGTGTTTTTCTTACTGGAGC | |
| TTCATTTTGGTAAGAGACAGAATGTATCTGTGAAGTAGTTCTGTTTGGTGTGTCCCATTGGTGTTGTCAT | |
| TGTAAACAAACTCTTGAAGAGTCGATTATTTCCAGTGTTCTATGAACAACTCCAAAACCCATGTGGGAAA | |
| AAAATGAATGAGGAGGGTAGGGAATAAAATCCTAAGACACAAATGCATGAACAAGTTTTAATGTATAGTT | |
| TTGAATCCTTTGCCTGCCTGGTGTGCCTCAGTATATTTAAACTCAAGACAATGCACCTAGCTGTGCAAGA | |
| CCTAGTGCTCTTAAGCCTAAATGCCTTAGAAATGTAAACTGCCATATATAACAGATACATTTCCCTCTTT | |
| CTTATAATACTCTGTTGTACTATGGAAAATCAGCTGCTCAGCAACCTTTCACCTTTGTGTATTTTTCAAT | |
| AATAAAAAATATTCTTGTCAAAAAAAAAAAAA | |

PRKACA mRNA GenBank Accession No. NM_{—}002730 (gi:4506054)

| | |
|---|---|
| CAGTGNGCTCCGGGCCGCCGGCCGCAGCCAGCACCCGCCGCGCCGCAGCTCCGGGACCGGCCCCGGCCGC | [SEQ ID NO: 116] |
| CGCCGCCGCGATGGGCAACGCCGCCGCCGCCAAGAAGGGCAGCGAGCAGGAGAGCGTGAAAGAATTCTTA | |
| GCCAAAGCCAAAGAAGATTTTCTTAAAAAATGGGAAAGTCCCGCTCAGAACACAGCCCACTTGGATCAGT | |
| TTGAACGAATCAAGACCCTCGGCACGGGCTCCTTCGGGCGGGTGATGCTGGTGAAACACAAGGAGACCGG | |
| GAACCACTATGCCATGAAGATCCTCGACAAACAGAAGGTGGTGAAACTGAAACAGATCGAACACACCCTG | |
| AATGAAAAGCGCATCCTGCAAGCTGTCAACTTTCCGTTCCTCGTCAAACTCGAGTTCTCCTTCAAGGACA | |
| ACTCAAACTTATACATGGTCATGGAGTACGTGCCCGGCGGGGAGATGTTCTCACACCTACGGCGGATCGG | |
| AAGGTTCAGTGAGCCCCATGCCCGTTTCTACGCGGCCCAGATCGTCCTGACCTTTGAGTATCTGCACTCG | |
| CTGGATCTCATCTACAGGGACCTGAAGCCGGAGAATCTGCTCATTGACCAGCAGGGCTACATTCAGGTGA | |
| CAGACTTCGGTTTCGCCAAGCGCGTGAAGGGCCGCACTTGGACCTTGTGCGGCACCCCTGAGTACCTGGC | |
| CCCTGAGATTATCCTGAGCAAAGGCTACAACAAGGCCGTGGACTGGTGGGCCCTGGGGGTTCTTATCTAT | |
| GAAATGGCCGCTGGCTACCCGCCCTTCTTCGCAGACCAGCCCATCCAGATCTATGAGAAGATCGTCTCTG | |
| GGAAGGTGCGCTTCCCTTCCCACTTCAGCTCTGACTTGAAGGACCTGCTGCGGAACCTCCTGCAGGTAGA | |
| TCTCACCAAGCGCTTTGGGAACCTCAAGAATGGGGTCAACGATATCAAGAACCACAAGTGGTTTGCCACA | |
| ACTGACTGGATTGCCATCTACCAGAGGAAGGTGGAAGCTCCCTTCATACCAAAGTTTAAAGGCCCTGGGG | |
| ATACGAGTAACTTTGACGACTATGAGGAAGAAGAAATCCGGGTCTCCATCAATGAGAAGTGTGGCAAGGA | |
| GTTTTCTGAGTTTTAGGGGCATGCCTGTGCCCCCATGGGTTTTTTTTTTTTTTTTTTTTTTTTTTTGGTC | |
| GGGGGGGTGGGAGGGTTGGATTGAACAGCCAGAGGGCCCCAGAGTTCCTTGCATCTAATTTCACCCCCAC | |
| CCCACCCTCCAGGGTTAGGGGGAGCAGGAAGCCCAGATAATCAGAGGGACAGAAACACCAGCTGCTCCCC | |
| CTCATCCCCTTCACCCTCCTGCCCCCTCTCCCACTTTTCCCTTCCTCTTTCCCCACAGCCCCCCAGCCCC | |
| TCAGCCCTCCCAGCCCACTTCTGCCTGTTTTAAACGAGTTTCTCAACTCCAGTCAGACCAGGTCTTGCTG | |
| GTGTATCCAGGGACAGGGTATGGAAAGAGGGGCTCACGCTTAACTCCAGCCCCCACCCACACCCCCATCC | |
| CACCCAACCACAGGCCCCACTTGCTAAGGGCAAATGAACGAAGCGCCAACCTTCCTTTCGGAGTAATCCT | |
| GCCTGGGAAGGAGAGATTTTTAGTGACATGTTCAGTGGGTTGCTTGCTAGAATTTTTTTAAAAAAACAAC | |
| AATTTAAAATCTTATTTAAGTTCCACCAGTGCCTCCCTCCCTCCTTCCTCTACTCCCACCCCTCCCATGT | |
| CCCCCCATTCCTCAAATCCATTTTAAAGAGAAGCAGACTGACTTTGGAAAGGGAGGCGCTGGGGTTTGAA | |
| CCTCCCCGCTGCTAATCTCCCCTGGGCCCCTCCCCGGGGAATCCTCTCTGCCAATCCTGCGAGGGTCTAG | |
| GCCCCTTTAGGAAGCCTCCGCTCTCTTTTTCCCCAACAGACCTGTCTTCACCCTTGGGCTTTGAAAGCCA | |
| GACAAAGCAGCTGCCCCTCTCCCTGCCAAAGAGGAGTCATCCCCCAAAAAGACAGAGGGGGAGCCCCAAG | |
| CCCAAGTCTTTCCTCCCAGCAGCGTTTCCCCCCAACTCCTTAATTTTATTCTCCGCTAGATTTTAACGTC | |
| CAGCCTTCCCTCAGCTGAGTGGGGAGGGCATCCCTGCAAAAGGGAACAGAAGAGGCCAAGTCCCCCCAAG | |
| CCACGGCCCGGGGTTCAAGGCTAGAGCTGCTGGGGAGGGGCTGCCTGTTTTACTCACCCACCAGCTTCCG | |
| CCTCCCCCATCCTGGGCGCCCCTCCTCCAGCTTAGCTGTCAGCTGTCCATCACCTCTCCCCCACTTTCTC | |
| ATTTGTGCTTTTTTCTCTCGTAATAGAAAAGTGGGGAGCCGCTGGGGAGCCACCCCATTCATCCCCGTAT | |
| TTCCCCCTCTCATAACTTCTCCCCATCCCAGGAGGAGTTCTCAGGCCTGGGGTGGGGCCCCGGGTGGGTG | |
| CGGGGGCGATTCAACCTGTGTGCTGCGAAGGACGAGACTTCCTCTTGAACAGTGTGCTGTTGTAAACATA | |
| TTTGAAAACTATTACCAATAAAGTTTGTT | |

ACVR1B mRNA GenBank Accession No. NM_{—}004302 (gi:10862695)

| | |
|---|---|
| CGCTGCTGGGCTGCGGCGGCGGCGGCGGCGGTGGTTACTATGGCGGAGTCGGCCGGAGCCTCCTCCTTCT | [SEQ ID NO: 117] |
| TCCCCCTTGTTGTCCTCCTGCTCGCCGGCAGCGGCGGGTCCGGGCCCCGGGGGGTCCAGGCTCTGCTGTG | |
| TGCGTGCACCAGCTGCCTCCAGGCCAACTACACGTGTGAGACAGATGGGGCCTGCATGGTTTCCATTTTC | |
| AATCTGGATGGGATGGAGCACCATGTGCGCACCTGCATCCCCAAAGTGGAGCTGGTCCCTGCCGGGAAGC | |
| CCTTCTACTGCCTGAGCTCGGAGGACCTGCGCAACACCCACTGCTGCTACACTGACTACTGCAACAGGAT | |
| CGACTTGAGGGTGCCCAGTGGTCACCTCAAGGAGCCTGAGCACCCGTCCATGTGGGGCCCGGTGGAGCTG | |
| GTAGGCATCATCGCCGGCCCGGTGTTCCTCCTGTTCCTCATCATCATCATTGTTTTCCTTGTCATTAACT | |
| ATCATCAGCGTGTCTATCACAACCGCCAGAGACTGGACATGGAAGATCCCTCATGTGAGATGTGTCTCTC | |
| CAAAGACAAGACGCTCCAGGATCTTGTCTACGATCTCTCCACCTCAGGGTCTGGCTCAGGGTTACCCCTC | |
| TTTGTCCAGCGCACAGTGGCCCGAACCATCGTTTTACAAGAGATTATTGGCAAGGGTCGGTTTGGGGAAG | |
| TATGGCGGGGCCGCTGGAGGGGTGGTGATGTGGCTGTGAAAATATTCTCTTCTCGTGAAGAACGGTCTTG | |
| GTTCAGGGAAGCAGAGATATACCAGACGGTCATGCTGCGCCATGAAAACATCCTTGGATTTATTGCTGCT | |
| GACAATAAAGATAATGGCACCTGGACACAGCTGTGGCTTGTTTCTGACTATCATGAGCACGGGTCCCTGT | |
| TTGATTATCTGAACCGGTACACAGTGACAATTGAGGGGATGATTAAGCTGGCCTTGTCTGCTGCTAGTGG | |
| GCTGGCACACCTGCACATGGAGATCGTGGGCACCCAAGGGAAGCCTGGAATTGCTCATCGAGACTTAAAG | |
| TCAAAGAACATTCTGGTGAAGAAAAATGGCATGTGTGCCATAGCAGACCTGGGCCTGGCTGTCCGTCATG | |
| ATGCAGTCACTGACACCATTGACATTGCCCCGAATCAGAGGGTGGGGACCAAACGATACATGGCCCCTGA | |
| AGTACTTGATGAAACCATTAATATGAAACACTTTGACTCCTTTAAATGTGCTGATATTTATGCCCTCGGG | |
| CTTGTATATTGGGAGATTGCTCGAAGATGCAATTCTGGAGGAGTCCATGAAGAATATCAGCTGCCATATT | |
| ACGACTTAGTGCCCTCTGACCCTTCCATTGAGGAAATGCGAAAGGTTGTATGTGATCAGAAGCTGCGTCC | |
| CAACATCCCCAACTGGTGGCAGAGTTATGAGGCACTGCGGGTGATGGGGAAGATGATGCGAGAGTGTTGG | |
| TATGCCAACGGCGCAGCCCGCCTGACGGCCCTGCGCATCAAGAAGACCCTCTCCCAGCTCAGCGTGCAGG | |
| AAGACGTGAAGATCTAACTGCTCCCTCTCTCCACACGGAGCTCCTGGCAGCGAGAACTACGCACAGCTGC | |
| CGCGTTGAGCGTACGATGGAGGCCTACCTCTCGTTTCTGCCCAGCCCTCTGTGGCCAGGAGCCCTGGCCC | |
| GCAAGAGGGACAGAGCCCGGGAGAGACTCGCTCACTCCCATGTTGGGTTTGAGACAGACACCTTTTCTAT | |
| TTACCTCCTAATGGCATGGAGACTCTGAGAGCGAATTGTGTGGAGAACTCAGTGCCACACCTCGAACTGG | |
| TTGTAGTGGGAAGTCCCGCGAAACCCGGTGCATCTGGCACGTGGCCAGGAGCCATGACAGGGGCGCTTGG | |
| TTGTAGTGGGAAGTCCCGCGAAACCCGGTGCATCTGGCACGTGGCCAGGAGCCATGACAGGGGCGCTTGG | |
| GAGGGGCCGGAGGAACCGAGGTGTTGCCAGTGCTAAGCTGCCCTGAGGGTTTCCTTCGGGGACCAGCCCA | |
| CAGCACACCAAGGTGGCCCGGAAGAACCAGAAGTGCAGCCCCTCTCACAGGCAGCTCTGAGCCGCGCTTT | |
| CCCCTCCTCCCTGGGATGGACGCTGCCGGGAGACTGCCAGTGGAGACGGAATCTGCCGCTTTGTCTGTCC | |
| AGCCGTGTGTGCATGTGCCGAGGTGCGTCCCCCGTTGTGCCTGGTTCGTGCCATGCCCTTACACGTGCGT | |
| GTGAGTGTGTGTGTGTGTCTGTAGGTGCGCACTTACCTGCTTGAGCTTTCTGTGCATGTGCAGGTCGGGG | |
| GTGTGGTCGTCATGCTGTCCGTGCTTGCTGGTGCCTCTTTTCAGTAGTGAGCAGCATCTAGTTTCCCTGG | |
| TGCCCTTCCCTGGAGGTCTCTCCCTCCCCCAGAGCCCCTCATGCCACAGTGGTACTCTGTGT | |

CDK5R1 mRNA, GenBank Accession No: NM_{—}003885 (gi:4502736)

| | |
|---|---|
| AAACTCAGAATTTTCGCGGGCTCGGTGAGCGGTTTTATCCCTCCGGCCGGCAGGCTGGCCGCAGGGGGCG | [SEQ ID NO: 118] |
| AGCCCCCGCCCGGCGCGCAGCAGCACCATGGGCACGGTGCTGTCCCTGTCTCCCAGCTACCGGAAGGCCA | |
| CGCTGTTTGAGGATGGCGCGGCCACCGTGGGCCACTATACGGCCGTACAGAACAGCAAGAACGCCAAGGA | |
| CAAGAACCTGAAGCGCCACTCCATCATCTCCGTGCTGCCTTGGAAGAGAATCGTGGCCGTGTCGGCCAAG | |
| AAGAAGAACTCCAAGAAGGTGCAGCCTAACAGCAGCTACCAGAACAACATCACGCACCTCAACAATGAGA | |
| ACCTGAAGAAGTCGCTGTCGTGCGCCAACCTGTCCACATTCGCCCAGCCCCCACCGGCCCAGCCGCCTGC | |
| ACCCCCGGCCAGCCAGCTCTCGGGTTCCCAGACCGGGGGCTCCTCCTCAGTCAAGAAAGCCCCTCACCCT | |
| GCCGTCACCTCCGCAGGGACGCCCAAACGGGTCATCGTCCAGGCGTCCACCAGTGAGCTGCTTCGCTGCC | |
| TGGGTGAGTTTCTCTGCCGCCGGTGCTACCGCCTGAAGCACCTGTCCCCCACGGACCCCGTGCTCTGGCT | |
| GCGCAGCGTGGACCGCTCGCTGCTTCTGCAGGGCTGGCAGGACCAGGGCTTCATCACGCCGGCCAACGTG | |
| GTCTTCCTCTACATGCTCTGCAGGGATGTTATCTCCTCCGAGGTGGGCTCGGATCACGAGCTCCAGGCCG | |
| TCCTGCTGACATGCCTGTACCTCTCCTACTCCTACATGGGCAACGAGATCTCCTACCCGCTCAAGCCCTT | |
| CCTGGTGGAGAGCTGCAAGGAGGCCTTTTGGGACCGTTGCCTCTCTGTCATCAACCTCATGAGCTCAAAG | |
| ATGCTGCAGATAAATGCCGACCCACACTACTTCACACAGGTCTTCTCCGACCTGAAGAACGAGAGCGGCC | |
| AGGAGGACAAGAAGCGGCTCCTCCTAGGCCTGGATCGGTGAGCACTGTAGCCTGCGTCATGGCTCAAGGA | |
| TTCAATGCATTTTTAAGAATTTATTATTAAATCAGTTTTGTGTACAG | |

CDC42BPB mRNA, GenBank Accession No.: NM_{—}006035.2 (gi:16357473)

| | |
|---|---|
| GGGCGGGGCTGAGGGCGGCGGGGGCGGGCCGCCCGAGCTGGGAGGGCGGCGGCGCCGAGGGGAGGAGAGC | [SEQ ID NO: 119] |
| GGCCCATGGACCCGCGGGGCCCGGCGCCCCACACTCTGCGCCGTCGGGACGGAGCCCAAGATGTCGGCCT | |
| AGGCCGGGGCGCGACGACGCGGACGGGGCGGCGAGGAGGCGCCGCTGCTGCCGGGGCTCGCAGCCGCCGA | |
| GCCCCCGAGGGCGCGCCCTGACGGACTGGCCGAGCCGGCGGTGAGAGGCCGGCGCGTCGGGAGCGGGCCG | |
| CGCGGCACCATGTCGGCCAAGGTGCGGCTCAAGAAGCTGGAGCAGCTGCTCCTGGACGGGCCCTGGCGCA | |
| ACGAGAGCGCCCTGAGCGTGGAAACGCTGCTCGACGTGCTCGTCTGCCTGTACACCGAGTGCAGCCACTC | |
| GGCCCTGCGCCGCGACAAGTACGTGGCCGAGTTCCTCGAGTGGGCTAAACCATTTACACAGCTGGTGAAA | |
| GAAATGCAGCTTCATCGAGAAGACTTTGAAATAATTGGAGTAATTGGAAGAGGTGCTTTTGGTGAGGTTG | |
| CTGTTGTCAAAATGAAGAATACTGAACGAATTTATGCAATGAAAATCCTCAACAAGTGGGAGATGCTGAA | |
| AAGAGCAGAGACCGCGTGCTTCCGAGAGGAGCGCGATGTGCTGGTGAACGGCGACTGCCAGTGGATCACC | |
| GCGCTGCACTACGCCTTTCAGGACGAGAACCACCTGTACTTAGTCATGGATTACTATGTGGGTGGTGATT | |
| TACTGACCCTGCTCAGCAAATTTGAAGACAAGCTTCCGGAAGATATGGCGAGGTTCTACATTGGTGAAAT | |
| GGTGCTGGCCATTGACTCCATCCATCAGCTTCATTACGTGCACAGAGACATTAAACCTGACAATGTCCTT | |
| TTGGACGTGAATGGTCATATCCGCCTGGCTGACTTTGGATCATGTTTGAAGATGAATGATGATGGCACTG | |
| TGCAGTCCTCCGTGGCCGTGGGCACACCTGACTACATCTCGCCGGAGATCCTGCAGGCGATGGAGGACGG | |
| CATGGGCAAATACGGGCCTGAGTGTGACTGGTGGTCTCTGGGTGTCTGCATGTATGAGATGCTCTATGGA | |
| GAAACGCCGTTTTATGCGGAGTCACTCGTGGAGACCTATGGGAAGATCATGAACCATGAAGAGCGATTCC | |
| AGTTCCCATCCCATGTCACGGATGTATCTGAAGAAGCGAAGGACCTCATCCAGAGACTGATCTGCAGTAG | |
| AGAACGCCGGCTGGGGCAGAATGGAATAGAGGATTTCAAAAAGCATGCGTTTTTTGAAGGTCTAAATTGG | |
| GAAAATATACGAAACCTAGAAGCACCTTATATTCCTGATGTGAGCAGTCCCTCTGACACATCCAACTTCG | |
| ACGTGGATGACGACGTGCTGAGAAACACGGAAATATTACCTCCTGGTTCTCACACAGGCTTTTCTGGATT | |
| ACATTTGCCATTCATTGGTTTTACATTCACAACGGAAAGCTGTTTTTCTGATCGAGGCTCTCTGAAGAGC | |
| ATAATGCAGTCCAACACATTAATTAAAGATGAGGATGTGCAGCGGGACCTGGAGCACAGCCTGCAGATGG | |
| AAGCTTACGAGAGGAGGATTCGGAGGCTGGAACAGGAGAAGCTGGAGCTGAGCAGGAAGCTGCAAGAGTC | |
| CACCCAGACCGTGCAGTCCCTCCACGGCTCATCTCGGGCCCTCAGCAATTCAAACCGAGATAAAGAAATC | |
| AAAAAGCTAAATGAAGAAATCGAACGCTTGAAGAATAAAATAGCAGATTCAAACAGGCTCGAGCGACAGC | |
| TTGAGGACACAGTGGCGCTTCGCCAAGAGCGTGAGGACTCCACGCAGCGGCTGCGGGGGCTGGAGAAGCA | |
| GCACCGCGTGGTCCGGCAGGAGAAGGAGGAGCTGCACAAGCAACTGGTTGAAGCCTCAGAGCGGTTGAAA | |
| TCCCAGGCCAAGGAACTCAAAGATGCCCATCAGCAGCGAAAGCTGGCCCTGCAGGAGTTCTCGGAGCTGA | |
| ACGAGCGCATGGCAGAGCTCCGTGCCCAGAAGCAGAAGGTGTCCCGGCAGCTGCGAGACAAGGAGGAGGA | |
| GATGGAGGTGGCCACGCAGAAGGTGGACGCCATGCGGCAGGAAATGCGGAGAGCTGAGAAGCTCAGGAAA | |
| GAGCTGGAAGCTCAGCTTGATGATGCTGTTGCTGAGGCCTCCAAGGAGCGCAAGCTTCGTGAGCACAGCG | |
| AGAACTTCTGCAAGCAAATGGAAAGCGAGCTGGAGGCCCTCAAGGTGAAGCAAGGAGGCCGGGGAGCGGG | |
| TGCCACCTTAGAGCACCAGCAAGAGATTTCCAAAATCAAATCCGAGCTGGAGAAGAAAGTCTTATTTTAT | |
| GAAGAGGAATTGGTCAGACGTGAGGCCTCCCATGTGCTAGAAGTGAAAAATGTGAAGAAGGAGGTGCATG | |
| ATTCAGAAAGCCACCAGCTGGCCCTGCAGAAAGAAATCTTGATGTTAAAAGATAAGTTAGAAAAGTCAAA | |
| GCGAGAACGGCATAACGAGATGGAGGAGGCAGTAGGTACAATAAAAGATAAATACGAACGAGAAAGAGCG | |
| ATGCTGTTTGATGAAAACAAGAAGCTAACTGCTGAAAATGAAAAGCTCTGTTCCTTTGTGGATAAACTCA | |
| CAGCTCAAAATAGACAGCTGGAGGATGAGCTGCAGGATCTGGCAGCCAAGAAGGAGTCAGTGGCCCACTG | |
| GGAAGCTCAGATTGCGGAAATCATTCAGTGGGTCAGTGACGAGAAAGATGCCCGGGGTTACCTTCAAGCT | |
| CTTGCTTCCAAGATGACCGAAGAGCTCGAGGCTTTGAGGAGTTCTAGTCTGGGGTCAAGAACACTGGACC | |
| CGCTGTGGAAGGTGCGCCGCAGCCAGAAGCTGGACATGTCCGCGCGGCTGGAGCTGCAGTCGGCCCTGGA | |
| GGCGGAGATCCGGGCCAAGCAGCTTGTCCAGGAGGAGCTCAGGAAGGTCAAGGACGCCAACCTCACCTTG | |
| GAAAGCAAACTAAAGGATTCCGAAGCCAAAAACAGAGAATTATTAGAAGAAATGGAAATTTTGAAGAAAA | |
| AGATGGAAGAAAAATTCAGAGCAGATACTGGGCTCAAACTTCCAGATTTTCAGGATTCCATTTTTGAGTA | |
| TTTCAACACTGCTCCTCTTGCACATGACCTGACATTTAGAACCAGCTCAGCTAGTGAGCAAGAAACACAA | |
| GCTCCGAAGCCAGAAGCGTCCCCGTCGATGTCTGTGGCTGCATCAGAGCAGCAGGAGGACATGGCTCCGC | |
| CCCCGCAGAGGCCATCCGCTGTGCCGTTGCCCACCACGCAGGCCCTGGCTCTGGCTGGACCGAAGCCAAA | |
| AGCTCACCAGTTCAGCATCAAGTCCTTCTCCAGCCCTACTCAGTGCAGCCACTGCACCTCCCTGATGGTT | |
| GGGCTGATCCGGCAGGGCTACGCCTGCGACGTGTGTTCCTTTGCTTGCCACGTGTCCTGCAAAGACGGTG | |
| CCCCCCAGGTGTGCCCAATACCTCCCGAGCAGTCCAAGAGGCCTCTGGGCGTGGACGTGCAGCGAGGCAT | |
| CGGAACAGCCTACAAAGGCCATGTCAAGGTCCCAAAGCCCACGGGGGTGAAGAAGGGATGGCAGCGCGCA | |
| TATGCAGTCGTCTGTGAGTGCAAGCTCTTCCTGTATGATCTGCCTGAAGGAAAATCCACCCAGCCTGGTG | |
| TCATTGCGAGCCAAGTCTTGGATCTCAGAGATGACGAGTTTTCCGTGAGCTCAGTCCTGGCCTCAGATGT | |
| CATTCATGCTACACGCCGAGATATTCCATGTATATTCAGGGTGACGGCCTCTCTCTTAGGTGCACCTTCT | |
| AAGACCAGCTCGCTGCTCATTCTGACAGAAAATGAGAATGAAAAGAGGAAGTGGGTTGGGATTCTAGAAG | |
| GACTCCAGTCCATCCTTCATAAAAACCGGCTGAGGAATCAGGTCGTGCATGTTCCCTTGGAAGCCTACGA | |
| CAGCTCGCTGCCTCTCATCAAGGCCATCCTGACAGCTGCCATCGTGGATGCAGACAGGATTGCAGTCGGC | |
| CTAGAAGAAGGGCTCTATGTCATAGAGGTCACCCGAGATGTGATCGTCCGTGCCGCTGACTGTAAGAAGG | |
| TACACCAGATCGAGCTTGCTCCCAGGGAGAAGATCGTAATCCTCCTCTGTGGCCGGAACCACCATGTGCA | |
| CCTCTATCCGTGGTCGTCCCTTGATGGAGCGGAAGGCAGCTTTGACATCAAGCTTCCGGAAACCAAAGGC | |
| TGCCAGCTCATGGCCACGGCCACACTCAAGAGGAACTCTGGCACCTGCCTGTTTGTGGCCGTGAAACGGC | |
| TGATCCTTTGCTATGAGATCCAGAGAACGAAGCCATTCCACAGAAAGTTCAATGAGATTGTGGCTCCCGG | |
| CAGCGTGCAGTGCCTGGCGGTGCTCAGGGACAGGCTCTGTGTGGGCTACCCTTCTGGGTTCTGCCTGCTG | |
| AGCATCCAGGGGGACGGGCAGCCTCTAAACCTGGTAAATCCCAATGACCCCTCGCTTGCGTTCCTCTCAC | |
| AACAGTCTTTTGATGCCCTTTGTGCTGTGGAGCTCGAAAGCGAGGAGTACCTGCTTTGCTTCAGCCACAT | |
| GGGACTGTACGTGGACCCGCAAGGCCGGAGGGCACGCGCGCAGGAGCTCATGTGGCCTGCGGCTCCTGTC | |
| GCCTGTAGTTGCAGCCCCACCCACGTCACGGTGTACAGCGAGTATGGCCTGGACGTCTTTGATGTGCGCA | |
| CCATGGAGTGGGTGCAGACCATCGGCCTGCGGAGGATAAGGCCCCTGAACTCTGAAGGCACCCTCAACCT | |
| CCTCAACTGCGAGCCTCCACGCTTGATCTACTTCAAGAGCAAGTTCTCGGGAGCGGTTCTCAACGTGCCG | |
| GACACCTCCGACAACAGCAAGAAGCAGATGCTGCGCACCAGGAGCAAAAGGCGGTTCGTCTTCAAGGTCC | |
| CAGAGGAAGAGAGACTGCAGCAGAGGCGAGAGATGCTTAGAGACCCAGAATTGAGATCCAAAATGATATC | |
| CAACCCAACCAACTTCAACCACGTGGCCCACATGGGCCCAGGCGACGGCATGCAGGTGCTCATGGACCTG | |
| CCTCTGAGTGCTGTGCCCCCCTCCCAGGAGGAAAGGCCGGGCCCCGCTCCCACCAACCTGGCTCGCCAGC | |
| CTCCATCCAGGAACAAGCCCTACATCTCGTGGCCCTCATCAGGTGGATCGGAGCCTAGCGTGACTGTGCC | |
| TCTGAGAAGTATGTCTGATCCAGACCAGGACTTTGACAAAGAGCCTGATTCGGACTCCACCAAACACTCA | |
| ACTCCATCGAATAGCTCCAACCCCAGCGGCCCACCGAGCCCCAACTCCCCCCACAGGAGCCAGCTCCCCC | |
| TCGAAGGCCTGGAGCAGCCGGCCTGTGACACCTGAAGCCGCCAGCTCGCCACAGGGGCCAGGGAGCTGGA | |
| GATGGCCTCCAGCGTCAGTGCCAAGACTGAGCGGGCCCTCCAGTGTTGTCCAAGGAAATGTAGAATCACT | |
| TTGTAGATATGGAGATGAAGAAGACAAATCTTTATTATAATATTGATCAGTTTTATGCCGCATTGTTCGT | |
| GGCAGTAGACCACATCTGTTCGTCTGCACAGCTGTGAGGCGATGCTGTTCCATCTGCACATGAAGGACCC | |
| CCATACAGCCTGTCTCCCACCCCTGACAACCCGAGAGGGCATATGGGGCCCTGCCAACACCACTTCCTCA | |
| GCAGAAACCCGTCATGACGCGGCTGCTTCGGAAGCAGACATCTGGGGACACAGCCTCAGTACCCAGTCTT | |
| TTCCCTAGTTCCTGAAACTTTCCTAGGACCTTAAGAGAATAGTAGGAGGTCCTATAGCATTCCCAGTGTC | |
| ACTAGAATTTTGAAGACAGGAAAGTGGAGGTTAGTCTGTGGCCTTTTTTTCATTTAGCCATTGCACAGTC | |
| AGCTGCAGAAGTCCTGCTGACCACCTAGTCATGGACAAAGGCCCAGGACCAGTGACACCCTGCGTCCCTG | |
| TGTGCATTAAGTTCATTCTGGGTCGCAGCCATGAAGTGTCACCAGTATCTACTACTGTGAAGTCAGCTGT | |
| GCTGTTTTCCATTCGCTTCCACGGCTTCTGCCTCCTGCCATAAAACCAGCGAGTGTCGTGGTGCAGGCAG | |
| GCCCTGTGGCCTGCTGGGCTGAGGGAAGTCAGAGCCCCAGGGCGCCACGAAGCAGCCACTGGGATACCCC | |
| ACCCCGCCCCGCCCTGCCCCCCCCCCCCCCCACCAGTCCTGCCCCCGCATGGAGCCCCCGTGATTAGTAG | |
| CCCGTATGATCACGTAGACCCACCCAACACACTCCTGCACACTGGCCCCGGCCCACGGCACAGCAATCCC | |
| CTGCGCGTGGATTTCACCTCACCCTTTGTACCAGATGTTGAGTGACCAGCTCTGTGGCCCTGTGTCGTCA | |
| GAGGCTTGTGATTAACTGTGGCGGCAGACACAGCTTGTCCACAGCTTGGGCCAGGCTTCCCCTGTCCTCC | |
| CACCGGTCGCCTGCTTGGCAAGGCTGTTCAGGACGTGCACTTCCCCAAGTCGGCACTGAGTGGCCCAGCA | |
| CCGCCTAGCCCTGCCACCCCACTGCCCTCCTGGGCCTTCTGCTGGATGGGCACCTGGGGGGTTCTGGTTT | |
| TTACTTTTTTAATGTAAGTCTCAGTCTTTGTAATTAATTATTGAATTGTGAGAACATTTTTGAACAATTT | |
| ACCTGTCAATAAAGCAGAAGACGGCAGTTTTAAAGTTAAAAAAAAAAAAAAAAAAAAAAAAA | |
| CAACTACGAGCCACGAGTTTGCAGATGGGGCTGCTCGGCGGCGCCTGTGGCTGAGGGAGAGCAGCGGCGG | [SEQ ID NO: 120] |
| CGGGGAGCGACCGGGAGCGGCGGCAGCGGCGGCGCGGAGGCGGCTGAGGTGCGAGCCGGACTAAATCATT | |
| TTGCTACTTTAAAAAAATCACGAAAGTACATTATTTGAAGTTTGGAGAAGAAAGGGATTTGGTAACAAAG | |
| GACAGCCATTTCCATTTTAAGCAGCTAAACAGCAGGAGAGATTTCTGTAAGAAGGTACCAGCTCAGATTC | |
| CATTGTTCATCATTTTGCAATGCAGCAAGTCTTGGAAAACCTTACGGAGCTGCCCTCGTCTACTGGAGCA | |
| GAAGAAATAGACCTAATTTTCCTCAAGGGAATTATGGAGAATCCTATTGTAAAATCACTTGCTAAGGCTC | |
| ATGAGAGGCTAGAAGATTCCAAACTAGAAGCTGTCAGTGACAATAACTTGGAATTAGTCAATGAAATTCT | |
| TGAAGACATCACTCCTCTAATAAATGTGGATGAAAATGTGGCAGAATTGGTTGGTATACTCAAAGAACCT | |
| CACTTCCAGTCACTGTTGGAGGCCCATGATATTGTGGCATCAAAGTGTTATGATTCACCTCCATCAAGCC | |
| CAGAAATGAATAATTCTTCTATCAATAATCAGTTATTACCAGTAGATGCCATTCGTATTCTTGGTATTCA | |
| CAAAAGAGCTGGGGAACCACTGGGTGTGACATTTAGGGTTGAAAATAATGATCTGGTAATTGCCCGAATC | |
| CTCCATGGGGGAATGATAGATCGACAAGGTCTACTTCATGTGGGAGATATAATTAAAGAAGTCAATGGCC | |
| ATGAGGTTGGAAATAATCCAAAGGAATTACAAGAATTACTGAAAAATATTAGTGGAAGTGTCACCCTAAA | |
| AATCTTACCAAGTTATAGAGATACCATTACTCCTCAACAGGTATTTGTGAAGTGTCATTTTGATTATAAT | |
| CCATACAATGACAACCTAATACCTTGCAAAGAAGCAGGATTGAAGTTTTCCAAAGGAGAAATTCTTCAGA | |
| TTGTAAATAGAGAAGATCCAAATTGGTGGCAGGCTAGCCATGTAAAAGAGGGAGGAAGCGCTGGTCTCAT | |
| TCCAAGCCAGTTCCTGGAAGAGAAGAGAAAGGCATTTGTTAGAAGAGACTGGGACAATTCAGGACCTTTT | |
| TGTGGAACTATAAGTAGCAAAAAAAAGAAAAAGATGATGTATCTCACAACCAGAAATGCAGAATTTGATC | |
| GTCATGAAATCCAGATATATGAGGAGGTAGCCAAAATGCCTCCCTTCCAGAGAAAAACATTAGTATTGAT | |
| AGGAGCTCAAGGTGTAGGCCGAAGAAGCTTGAAAAACAGGTTCATAGTATTGAATCCCACTAGATTTGGA | |
| ACTACGGTGCCATTTACTTCACGGAAACCAAGGGAAGATGAAAAAGATGGCCAGGCATATAAGTTTGTGT | |
| CACGATCTGAGATGGAAGCAGATATTAAAGCTGGAAAGTATTTGGAACATGGGGAATATGAAGGAAATCT | |
| CTATGGAACCAAAATTGATTCTATTCTTGAGGTTGTCCAAACTGGACGGACTTGCATTCTGGATGTCAAC | |
| CCACAAGCACTGAAAGTATTGAGGACATCAGAGTTTATGCCCTATGTGGTATTTATTGCGGCTCCGGAGC | |
| TAGAGACGTTACGTGCCATGCACAAGGCTGTGGTGGATGCAGGAATCACTACCAAGCTTCTGACCGACTC | |
| TGACTTGAAGAAAACAGTGGATGAAAGTGCACGGATTCAGAGAGCATACAACCACTATTTTGATTTGATC | |
| ATCATAAATGATAATCTAGACAAAGCCTTTGAAAAACTGCAAACTGCCATAGAGAAACTGAGAATGGAAC | |
| CACAGTGGGTCCCAATCAGCTGGGTTTACTGATGATTCAGTAAGGTTAACAATGAAAATTAAACTCTTAA | |
| AAAGTGACTGCAACAAATAAACCTTCTACTGAGAAAATACATCACAGATAGAAGATTATCTGCTAAGTCC | |
| AGGCATTTTTATGGTGTAGATTGAAATAATAGTACACTTCTGAATTTTTATATAAAATGTGGTTGGAAGG | |
| TGTACTAATATATAATTTATCTTAATTTTTCTAACTTTGTATGGATAATCTTTCTATTCATATCACATAA | |
| AGAAATGCGTTGAAGCAAAAAAAAAAAAAAA | |

MPP6 mRNA, GenBank Accession No.: NM_{—}016447 (gi:21361597)

CDC42BPA mRNA, GenBank Accession No.: NM_{—}014826 (gi:28274696)

| | |
|---|---|
| ATGTCTGGAGAAGTGCGTTTGAGGCAGTTGGAGCAGTTTATTTTGGACGGGCCCGCTCAGACCAATGGGC | [SEQ ID NO: 121] |
| AGTGCTTCAGTGTGGAGACGTTACTGGATATACTCATCTGCCTTTATGATGAATGCAATAATTCTCCATT | |
| GAGAAGAGAGAAGAACATTCTCGAATACCTAGAATGGGCTAAACCATTTACTTCTAAAGTGAAACAAATG | |
| CGATTACATAGAGAAGACTTTGAAATATTAAAGGTGATTGGTCGAGGAGCTTTTGGGGAGGTTGCTGTAG | |
| TAAAACTAAAAAATGCAGATAAAGTGTTTGCCATGAAAATATTGAATAAATGGGAAATGCTGAAAAGAGC | |
| TGAGACAGCATGTTTTCGTGAAGAAAGGGATGTATTAGTGAATGGAGACAATAAATGGATTACAACCTTG | |
| CACTATGCTTTCCAGGATGACAATAACTTATACCTGGTTATGGATTATTATGTTGGTGGGGATTTGCTTA | |
| CTCTACTCAGCAAATTTGAAGATAGATTGCCTGAAGATATGGCTAGATTTTACTTGGCTGAGATGGTGAT | |
| AGCAATTGACTCAGTTCATCAGCTACATTATGTACACAGAGACATTAAACCTGACAATATACTGATGGAT | |
| ATGAATGGACATATTCGGTTAGCAGATTTTGGTTCTTGTCTGAAGCTGATGGAAGATGGAACGGTTCAGT | |
| CCTCAGTGGCTGTAGGAACTCCAGATTATATCTCTCCTGAAATCCTTCAAGCCATGGAAGATGGAAAAGG | |
| GAGATATGGACCTGAATGTGACTGGTGGTCTTTGGGGGTCTGTATGTATGAAATGCTTTACGGAGAAACA | |
| CCATTTTATGCAGAATCGCTGGTGGAGACATACGGAAAAATCATGAACCACAAAGAGAGGTTTCAGTTTC | |
| CAGCCCAAGTGACTGATGTGTCTGAAAATGCTAAGGATCCTATTCGAAGGCTCATTTGTGGCAGAGAACA | |
| TCGACTTGGTCAAAGTGGAATAGAAGACTTTAAGAAACACCCATTTTTCAGTGGAATTGACTGGGATAAT | |
| ATTCGGAACTGTGAAGCACCTTATATTCCAGAAGTTAGTAGCCCAACAGATACATCGAATTTTGATGTAG | |
| ATGATGATTGTTTAAAAAATTCTGAAACGATGCCCCCACCAACACATACTGCATTTTCTGGCCACCATCT | |
| GCCATTTGTTGGTTTTACATATACTAGTAGCTGTGTACTTTCTGATCGGAGCTGTTTAAGAGTTACGGCT | |
| GGTCCCACCTCACTGGATCTTGATGTTAATGTTCAGAGGACTCTAGACAACAACTTAGCAACTGAAGCTT | |
| ATGAAAGAAGAATTAAGCGCCTTGAGCAAGAAAAACTTGAACTCAGTAGAAAACTTCAAGAGTCAACACA | |
| GACTGTCCAAGCTCTGCAGTATTCAACTGTTGATGGTCCACTAACAGCAAGCAAAGATTTAGAAATAAAA | |
| AACTTAAAAGAAGTAATTGAAAAACTAAGAAAACAAGTAACAGAATCAAGTCATTTGGAACAGCAACTTG | |
| AAGAAGCTAATGCTGTGAGGCAAGAACTAGATGATGCTTTTAGACAAATCAAGGCTTATGAAAAACAAAT | |
| CAAAACGTTACAACAAGAAAGAGAAGATCTAAATAAGCTGGAAGTTCATACAGAAGCTCTAGCTGCTGAA | |
| GCATCTAAAGACAGGAAGCTACGTGAACAGAGTGAGCACTATTCTAAGCAACTGGAAAATGAATTGGAGG | |
| GACTGAAGCAAAAACAAATTAGTTACTCACCAGGAGTATGCAGCATAGAACATCAGCAAGAGATAACCAA | |
| ACTAAAGACTGATTTGGAAAAGAAAAGTATCTTTTATGAAGAAGAATTATCTAAAAGAGAAGGAATACAT | |
| GCAAATGAAATAAAAAATCTTAAGAAAGAACTGCATGATTCAGAAGGTCAGCAACTTGCTCTCAACAAAG | |
| AAATTATGATTTTAAAAGACAAATTGGAAAAAACCAGAAGAGAAAGTCAAAGTGAAAGGGAGGAATTTGA | |
| AAGTGAGTTCAAACAACAATATGAACGAGAAAAAGTGTTGTTAACTGAAGAAAATAAAAAGCTGACGAGT | |
| GAACTTGATAAGCTTACTACTTTGTATGAGAACTTAAGTATACACAACCAGCAGTTAGAAGAAGAGGTTA | |
| AAGATCTAGCAGACAAGAAAGAATCAGTTGCACATTGGGAAGCCCAAATCACAGAAATAATTCAGTGGGT | |
| CAGCGATGAAAAGGATGCACGAGGGTATCTTCAGGCCTTAGCTTCTAAAATGACTGAAGAATTGGAGGCA | |
| TTAAGAAATTCCAGCTTGGGTACACGAGCAACAGATATGCCCTGGAAAATGCGTCGTTTTGCGAAACTGG | |
| ATATGTCAGCTAGACTGGAGTTGCAGTCGGCTCTGGATGCAGAAATAAGAGCCAAACAGGCCATCCAAGA | |
| AGAGTTGAATAAAGTTAAAGCATCTAATATCATAACAGAATGTAAACTAAAAGATTCAGAGAAGAAGAAC | |
| TTGGAACTACTCTCAGAAATCGAACAGCTGATAAAGGACACTGAAGAGCTTAGATCTGAAAAGGGTATAG | |
| AGCACCAAGACTCACAGCATTCTTTCTTGGCATTTTTGAATACGCCTACCGATGCTCTGGATCAATTTGA | |
| AACTGTAGACTCCACTCCACTTTCAGTTCACACACCAACCTTAAGGAAAAAAGGATGTCCTGGTTCAACT | |
| GGCTTTCCACCTAAGCGCAAGACTCACCAGTTTTTTGTAAAATCTTTTACTACTCCTACCAAGTGTCATC | |
| AGTGTACCTCCTTGATGGTGGGTTTAATAAGACAGGGCTGTTCATGTGAAGTGTGTGGATTCTCATGCCA | |
| TATAACTTGTGTAAACAAAGCTCCAACCACTTGTCCAGTTCCTCCTGAACAGACAAAAGGTCCCCTGGGT | |
| ATAGATCCTCAGAAAGGAATAGGAACAGCATATGAAGGTCATGTCAGGATTCCTAAGCCAGCTGGAGTGA | |
| AGAAAGGGTGGCAGAGAGCACTGGCTATAGTGTGTGACTTCAAACTCTTTCTGTACGATATTGCTGAAGG | |
| AAAAGCATCTCAGCCCAGTGTTGTCATTAGTCAAGTGATTGACATGAGGGATGAAGAATTTTCTGTGAGT | |
| TCAGTCTTGGCTTCTGATGTTATCCATGCAAGTCGGAAAGATATACCCTGTATATTTAGGGTCACAGCTT | |
| CCCAGCTCTCAGCATCTAATAACAAATGTTCAATCCTGATGCTAGCAGACACTGAGAATGAGAAGAATAA | |
| GTGGGTGGGAGTGCTGAGTGAATTGCACAAGATTTTGAAGAAAAACAAATTCAGAGACCGCTCAGTCTAT | |
| GTTCCCAAAGAGGCTTATGACAGCACTCTACCCCTCATTAAAACAACCCAGGCAGCCGCAATCATAGATC | |
| ATGAAAGAATTGCTTTGGGAAACGAAGAAGGGTTATTTGTTGTACATGTCACCAAAGATGAAATTATTAG | |
| AGTTGGTGACAATAAGAAGATTCATCAGATTGAACTCATTCCAAATGATCAGCTTGTTGCTGTGATCTCA | |
| GGACGAAATCGTCATGTACGACTTTTTCCTATGTCAGCATTGGATGGGCGAGAGACCGATTTTTACAAGC | |
| TGTCAGAAACTAAAGGGTGTCAAACCGTAACTTCTGGAAAGGTGCGCCATGGAGCTCTCACATGCCTGTG | |
| TGTGGCTATGAAAAGGCAGGTCCTCTGTTATGAACTATTTCAGAGCAAGACCCGTCACAGAAAATTTAAA | |
| GAAATTCAAGTCCCATATAATGTCCAGTGGATGGCAATCTTCAGTGAACAACTCTGTGTGGGATTCCAGT | |
| CAGGATTTCTAAGATACCCCTTGAATGGAGAAGGAAATCCATACAGTATGCTCCATTCAAATGACCATAC | |
| ACTATCATTTATTGCACATCAACCAATGGATGCTATCTGCGCAGTTGAGATCTCCAGTAAAGAATATCTG | |
| CTGTGTTTTAACAGCATTGGGATATACACTGACTGCCAGGGCCGAAGATCTAGACAACAGGAATTGATGT | |
| GGCCAGCAAATCCTTCCTCTTGTTGTTACAATGCACCATATCTCTCGGTGTACAGTGAAAATGCAGTTGA | |
| TATCTTTGATGTGAACTCCATGGAATGGATTCAGACTCTTCCTCTCAAAAAGGTTCGACCCTTAAACAAT | |
| GAAGGATCATTAAATCTTTTAGGGTTGGAGACCATTAGATTAATATATTTCAAAAATAAGATGGCAGAAG | |
| GGGACGAACTGGTAGTACCTGAAACATCAGATAATAGTCGGAAACAAATGGTTAGAAACATTAACAATAA | |
| GCGGCGTTATTCCTTCAGAGTCCCAGAAGAGGAAAGGATGCAGCAGAGGAGGGAAATGCTACGAGATCCA | |
| GAAATGAGAAATAAATTAATTTCTAATCCAACTAATTTTAATCACATAGCACACATGGGTCCTGGAGATG | |
| GAATACAGATCCTGAAAGATCTGCCCATGAACCCTCGGCCTCAGGAAAGTCGGACAGTATTCAGTGGCTC | |
| AGTCAGTATTCCATCTATCACCAAATCCCGCCCTGAGCCAGGCCGCTCCATGAGTGCTAGCAGTGGCTTG | |
| TCAGCAAGGTCATCCGCACAGAATGGCAGCGCATTAAAGAGGGAATTCTCTGGAGGAAGCTACAGTGCCA | |
| AGCGGCAGCCCATGCCCTCCCCGTCAGAGGGCTCTTTGTCCTCCGGAGGCATGGACCAAGGAAGTGATGC | |
| CCCAGCGAGGGACTTTGACGGAGAGGACTCTGACTCTCCGAGGCATTCCACAGCTTCCAACAGTTCCAAC | |
| CTAAGCAGCCCCCCAAGCCCAGTTTCACCCCGAAAAACCAAGAGCCTCTCCCTGGAGAGCACTGACCGCG | |
| GGAGCTGGGACCCGTGA | |

The invention has been described with an emphasis on preferred embodiments, however, the ordinarily skilled artisan will recognize that variations of the preferred embodiments can be used and that is not limited to the particular embodiments described herein. Accordingly, this invention includes all modifications encompassed within the spirit and scope of the invention as defined by the following claims.

All of the references cited herein, including patents, patent applications, and references, are hereby incorporated in their entireties by reference to the same extent as if each reference cited herein were individually incorporated by reference.

What is claimed is:

1. A method of killing a cancer cell, the method comprising contacting the cancer cell with an inhibitor of a gene selected from the group consisting of CDK8, STK33, PRKCM, PRKACA, ACVR1B, CDK5R1, CDC42BPB, MPP6, and CDC42BPA.

2. The method of claim 1, wherein the inhibitor is contacted to the cancer cell in a sterile composition comprising a pharmaceutically acceptable carrier.

3. The method of claim 1, wherein the inhibitor is an siRNA.

4. A sterile pharmaceutical composition comprising a nucleic acid capable of inhibiting a gene selected from the group consisting of CDK8, STK33, PRKCM, PRKACA, ACVR1B, CDK5R1, CDC42BPB, MPP6, and CDC42BPA in a pharmaceutically acceptable carrier.

5. An inhibitor of the expression of a gene selected from the group consisting of CDK8, STK33, PRKCM, PRKACA, ACVR1B, CDK5R1, CDC42BPB, MPP6, and CDC42BPA comprising an oligonucleotide having a nucleotide sequence selected from the group consisting of SEQ ID NOS: 1-6 and 11-78.
